(19) United States
(12) Patent Application Publication
(10) Pub. No.: US 2022/0373398 A1
(43) Pub. Date: Nov. 24, 2022

(54) APPARATUS AND METHOD FOR MEASURING AND PROVIDING BODY TEMPERATURE OF USER

(71) Applicant: Samsung Electronics Co., Ltd., Gyeonggi-do (KR)

(72) Inventors: Seungwon LEE, Gyeonggi-do (KR); Jeongmin PARK, Gyeonggi-do (KR); Daehyeong LIM, Gyeonggi-do (KR); Injo JEONG, Gyeonggi-do (KR)

(21) Appl. No.: 17/826,319

(22) Filed: May 27, 2022

Related U.S. Application Data
(63) Continuation of application No. PCT/KR2022/007109, filed on May 18, 2022.

(30) Foreign Application Priority Data
May 21, 2021 (KR) 10-2021-0065285

(51) Int. Cl. G01K 1/04; G01K 1/143; A61B 5/00; G01K 1/024; A61B 5/01

(52) U.S. Cl. CPC G01K 1/045; G01K 1/143; A61B 5/0002; G01K 1/024; A61B 5/01; A61B 2562/0271

(57) ABSTRACT

According to certain embodiments, an electronic device comprises: a sensor module; a communication module; and a processor operatively connected to the sensor module and the communication module, wherein the processor is configured to: measure a body temperature of a user through the sensor module, thereby resulting in a measured body temperature, estimate a core body temperature of the user, based on the measured body temperature, and provide body temperature information of the user, based on the core body temperature, wherein the measured body temperature comprises a shell temperature of a body part making contact with the electronic device, and wherein the core body temperature comprises a core temperature of an internal organ of a body of the user.

### CROSS-REFERENCE TO RELATED APPLICATION(S)

This application is a continuation of International Application No. PCT/KR2022/007109 filed on May 18, 2022, which claims priority to Korean Patent Application No. 10-2021-0065285, filed on May 21, 2021 in the Korean Intellectual Property Office, the disclosures of which are herein incorporated by reference.

### TECHNICAL FIELD

Embodiments of the disclosure relate to a method and an apparatus for measuring and providing temperature of a user.

### BACKGROUND

An electronic device may include a sensor for sensing (or measuring) various conditions and providing sensing data regarding the conditions. Accordingly, the electronic device can provide data (or functions) based on sensing data acquired from the sensor. For example, the electronic device may take biometric measurement through the sensor and provide information (for example, health information) related to a user's health. The electronic device may include a sensor (for example, a body temperature sensor) capable of measuring temperature of a user and providing temperature may be provided through the electronic device. For example, the electronic device may measure temperature of a user's skin and provide temperature information to the user.

A wearer of an electronic device may wish to measure the temperature of another person, such as the wearer's child. Measurement of the temperature of the other person may be complicated by a number of factors. There may be spatial restrictions. There may be various elements (for example, food intake, surrounding temperature, and/or fever by diseases) influencing body temperature of the other person that are not known. For example, for people to be measured (for example, people to be measured existing outside) out of view of the other person, the other person cannot identify various elements influencing body temperature of the person to be measured. Accordingly, the other person may not consider various elements for body temperature information of the person to be measured, measured and provided by the electronic device of the person to be measured, and thus reliability of the measured body temperature information may be low. As a result, the measured temperature of the other person may not be very accurate.

Even in a situation where the other person is in proximity, if body temperature is measured using a scheme other than directed measurement, it is difficult to unambiguously measure the body temperature of the desired other person, as opposed to other persons that are also proximate.

### SUMMARY

According to certain embodiments, an electronic device comprises: a sensor module; a communication module; and a processor operatively connected to the sensor module and the communication module, wherein the processor is configured to: measure a body temperature of a user through the sensor module, thereby resulting in a measured body temperature, estimate a core body temperature of the user, based on the measured body temperature, and provide body temperature information of the user, based on the core body temperature, wherein the measured body temperature comprises a shell temperature of a body part making contact with the electronic device, and wherein the core body temperature comprises a core temperature of an internal organ of a body of the user.

An operation method performed by an electronic device (for example, the first electronic device 201 of FIG. 2) according to certain embodiments of the disclosure may include an operation of measuring body temperature of a user through a sensor module of the electronic device, thereby resulting in a measured body temperature, estimating core body temperature of the user, based on the measured body temperature, providing body temperature information of the user, based on the core body temperature, wherein the measured body temperature includes shell temperature of a body part making contact with the electronic device; and wherein the core body temperature includes the core temperature of an internal organ of the body of the user.

An operation method performed by an electronic device (for example, the second electronic device 101 of FIG. 3) according to certain embodiments of the disclosure may include an operation of searching for at least one neighboring external device, in response to a user input, an operation of providing a list, based on at least one found external device on a display module, an operation of detecting an event for acquiring body temperature information, an operation of acquiring body temperature information from a selected one of the at least one neighboring external device, and an operation of providing the body temperature information through a display module.

A method of operating an electronic device, the method comprising: searching for at least one neighboring external device in response to a user input; providing a list, based on at least one found external device on a display module; detecting an event for acquiring body temperature information; acquiring body temperature information from a selected one of the at least one neighboring external device; and providing the body temperature information through the display module.

In order to solve the above problem, certain embodiments of the disclosure may include a computer-readable recording medium that records a program causing a processor to perform the method.

An additional range of applicability of the disclosure may become clear from the following detailed description. However, since various modifications and changes within the idea and scope of the disclosure may be clearly understood by those skilled in the art, the detailed description and specific embodiments such as exemplary embodiments of the disclosure should be understood as only examples.

According to an electronic device and a method of operating the same according to various embodiments, it is possible to support immediate and convenient acquisition and identification of body temperature of a person to be measured through predetermined communication between electronic devices by a person to measure in an environment in which the person to measure and the person to be measured exist in a limited distance.

According to various embodiments, the person to measure can immediately acquire and identify body temperature information by accurately designating the person to be measured even in a remote distance without access to the person to be measured by the person to measure through at least one predetermined communication (for example, BLE and/or UWB) in a limited distance such as indoors. According to various embodiments, it is possible to improve usability and accuracy of provision of body temperature information using the electronic device.

Further, various effects directly or indirectly detected through the disclosure can be provided.

### BRIEF DESCRIPTION OF DRAWINGS

In connection with the description of drawings, the same or similar reference numerals can be used for the same or similar elements.

FIG. 1 is a block diagram illustrating an example electronic device in a network environment according to certain embodiments.

FIG. 2 schematically illustrates an example of a block diagram of a first electronic device according to an embodiment of the disclosure.

FIG. 3 schematically illustrates an example of a block diagram of a second electronic device according to an embodiment of the disclosure.

FIG. 4 illustrates an example of the operation for measuring body temperature of a person to be measured in a system including a first electronic device and a second electronic device according to an embodiment of the disclosure.

FIG. 5 is a flowchart illustrating a method of operating an electronic device according to an embodiment of the disclosure.

FIG. 6 illustrates an example of distribution of body temperature of the interior of the body and body temperature of the surface of the body.

FIG. 7 is a flowchart illustrating a method of operating an electronic device according to an embodiment of the disclosure.

FIG. 8 illustrates a body temperature period graph and examples of feature values according to an embodiment.

FIG. 9 is a flowchart illustrating a method of operating an electronic device according to an embodiment of the disclosure.

FIG. 10 is a flowchart illustrating a method of operating an electronic device according to an embodiment of the disclosure.

FIG. 11 illustrates an example of a reference condition configured in an external device by an electronic device according to an embodiment.

FIG. 12 is a flowchart illustrating a method of operating an electronic device according to an embodiment of the disclosure.

FIG. 13A illustrates an example of a user interface for providing body temperature information in an electronic device according to an embodiment of the disclosure.

FIG. 13B illustrates an example of a user interface for providing body temperature information in an electronic device according to an embodiment of the disclosure.

FIG. 14 illustrates an example of a user interface for providing body temperature information in an electronic device according to an embodiment of the disclosure.

FIG. 15 illustrates an example of the operation for performing predetermined communication between electronic devices according to an embodiment of the disclosure.

FIG. 16 illustrates an example of an operation for providing body temperature information through predetermined communication between electronic devices according to an embodiment of the disclosure.

FIG. 17 illustrates an example of an operation for providing body temperature information through predetermined communication between electronic devices according to an embodiment of the disclosure.

### DETAILED DESCRIPTION

Certain embodiments provide a method and an apparatus for supporting immediate and convenient acquisition and identification of body temperature of another person through predetermined communication between electronic devices within a limited distance.

Certain embodiments provide a method and an apparatus for measuring body temperature and collecting and managing body temperature information according thereto by an electronic device of a first user (for example, the person to be measured).

Certain embodiments provide a method and an apparatus for acquiring body temperature information of the first user from the electronic device of the first user by an electronic device of a second user (for example, the person to measure for identifying body temperature of the first user).

Certain embodiments provide a method and an apparatus for providing body temperature information related to the first user through predetermined communication between the electronic devices of the first user and the second user in a predetermined space.

According to an electronic device and a method of operating the same according to certain embodiments, it is possible to support immediate and convenient acquisition and identification of body temperature of a person to be measured through predetermined communication between electronic devices by a person to measure in an environment in which the person to measure and the person to be measured exist in a limited distance.

According to certain embodiments, the person to measure can immediately acquire and identify body temperature information by accurately designating the person to be measured even in a remote distance without access to the person to be measured by the person to measure through at least one predetermined communication (for example, BLE and/or UWB) in a limited distance such as indoors. According to certain embodiments, it is possible to improve usability and accuracy of provision of body temperature information using the electronic device.

Further, various effects directly or indirectly detected through the disclosure can be provided.

Electronic Device

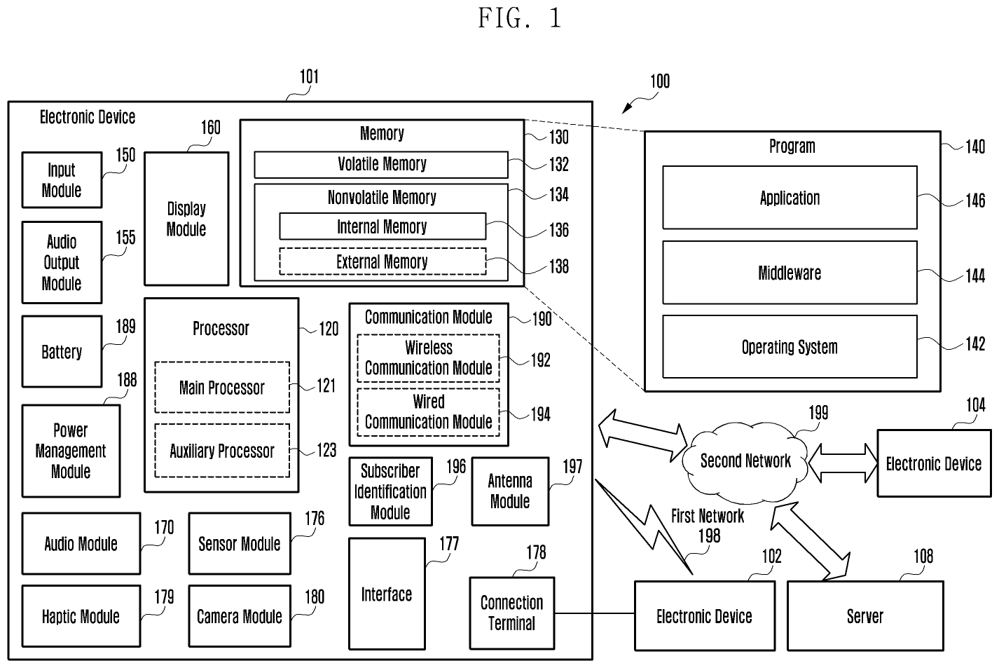

FIG. 1 describes one example of an electronic device. It shall be understood that electronic devices may omit, add, replace, or modify various aspects.

FIG. 1 is a block diagram illustrating an example electronic device 101 in a network environment 100 according to certain embodiments.

Referring to FIG. 1, the electronic device 101 in the network environment 100 may communicate with an electronic device 102 via a first network 198 (e.g., a short-range wireless communication network), or at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In certain embodiments, at least one of the components (e.g., the connecting terminal 178) may be omitted from the electronic device 101, or one or more other components may be added in the electronic device 101. In certain embodiments, some of the components (e.g., the sensor module 176, the camera module 180, or the antenna module 197) may be implemented as a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to an embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be adapted to consume less power than the main processor 121, or to be specific to a specified function. The auxiliary processor 123 may be implemented as separate from, or as part of the main processor 121.

The term “processor” shall be understood to refer to both the singular and plural context in this document.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to detect a touch, or a pressure sensor adapted to measure the intensity of force incurred by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, a SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to an embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a legacy cellular network, a 5G network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a 4G network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the mmWave band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device) of the electronic device 101. According to an embodiment, the antenna module 197 may include an antenna including a radiating element including a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to certain embodiments, the antenna module 197 may form a mmWave antenna module. According to an embodiment, the mmWave antenna module may include a printed circuit board, a RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In an embodiment, the external electronic device 104 may include an internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

The electronic device according to certain embodiments may be one of various types of electronic devices. The electronic devices may include, for example, a portable communication device (e.g., a smartphone), a computer device, a portable multimedia device, a portable medical device, a camera, a wearable device, a home appliance, or the like. According to an embodiment of the disclosure, the electronic devices are not limited to those described above.

It should be appreciated that certain embodiments of the disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), the element may be coupled with the other element directly (e.g., wiredly), wirelessly, or via a third element.

As used in connection with certain embodiments of the disclosure, the term “module” may include a unit implemented in hardware, software, or firmware, or any combination thereof, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Certain embodiments as set forth herein may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the “non-transitory” storage medium is a tangible device, and may not include a signal (e.g., an electromagnetic wave), but this term does not differentiate between where data is semi-permanently stored in the storage medium and where the data is temporarily stored in the storage medium.

According to an embodiment, a method according to certain embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to certain embodiments, each component (e.g., a module or a program) of the above-described components may include a single entity or multiple entities, and some of the multiple entities may be separately disposed in different components. According to certain embodiments, one or more of the above-described components may be omitted, or one or more other components may be added. Alternatively or additionally, a plurality of components (e.g., modules or programs) may be integrated into a single component. In such a case, according to certain embodiments, the integrated component may still perform one or more functions of each of the plurality of components in the same or similar manner as they are performed by a corresponding one of the plurality of components before the integration. According to certain embodiments, operations performed by the module, the program, or another component may be carried out sequentially, in parallel, repeatedly, or heuristically, or one or more of the operations may be executed in a different order or omitted, or one or more other operations may be added.

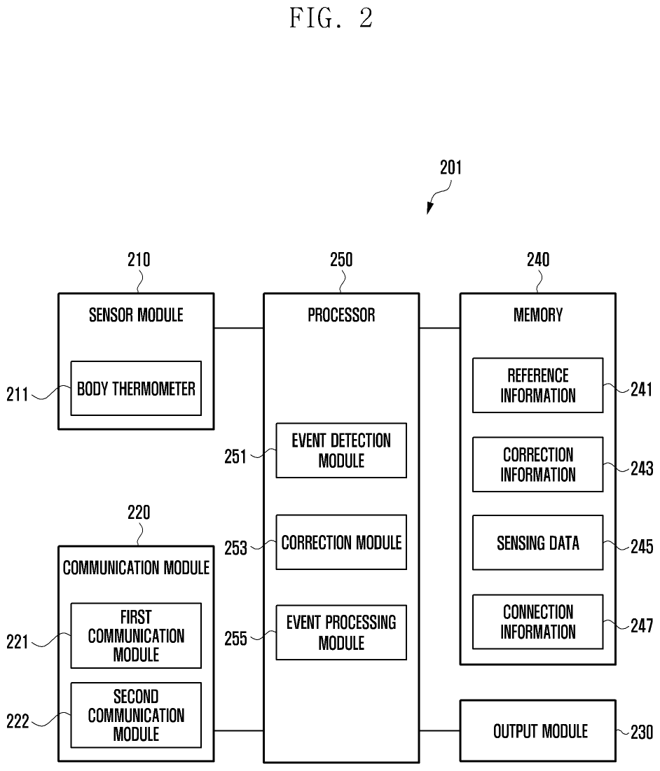

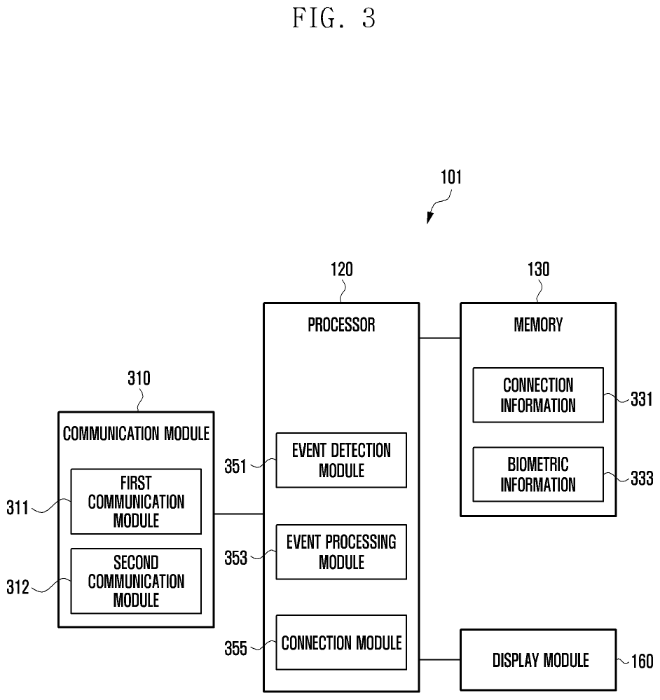

In certain embodiments, an electronic device 201 in FIG. 2 can be associated with the user whose temperature is to be taken, and another electronic device, e,g, the electronic device is FIG. 3 associated with another person, who may request the body temperature of the user of the electronic device 201.

Electronic Device of the Person Who is Measured

The electronic device 201 in response to a trigger or event, can take the body temperature of the user. The trigger or event can include, among other things, a request from an external electronic device via a communication module 220. The electronic device 201 includes a body temperature 211 that measures the body temperature of the user, by making physical contact with a body part of the user. The electronic device 201 estimates a core temperature of the user, based on the measured temperature. The core temperature of the user is the temperature of an internal organ. Based on the core temperature, the electronic device 201 can determine and provide body temperature information of the user.

FIG. 2 schematically illustrates an example of a block diagram of a first electronic device according to an embodiment of the disclosure.

In the disclosure, a first electronic device 201 according to an embodiment may indicate an electronic device of a person whose temperature (the subject) is to be measured. The first electronic device 201 is a device which can be worn on a body part of the subject, and may be a wearable device, for example, a watch type, a ring type, or an in-ear type device.

Referring to FIG. 2, the first electronic device 201 (for example, a wearable device) according to an embodiment may include a sensor module 210, a communication module 220, an output module 230, a memory 240, and/or a processor 250.

The sensor module 210 may include an element corresponding to the sensor module 176 as described with reference to FIG. 1. According to the disclosure, the sensor module 210 may include a body thermometer 211. Although not illustrated, the sensor module 210 may include various other sensors such as an acceleration sensor, an atmospheric pressure sensor, and/or a biometric sensor (for example, a PPG sensor). The first electronic device 201 may detect temperature of the user on the basis of sensing data using the body thermometer 211 of the sensor module 210.

The body thermometer 211 may be installed in the first electronic device 201 to detect temperature of the user in various designated schemes. For example, the body thermometer 211 may include various types of temperature sensors such as an infrared (IR) temperature sensor, an integrated circuit (IC) temperature sensor, a thermistor, or a resistance temperature detector (RTD).

The IR temperature sensor is a temperature sensor in an non-contact type, and may detect a degree of infrared ray radiated from the user's body, and filter and convert the same to estimate body temperature. The IC temperature sensor is the type using temperature dependency of a silicon band gap and may indicate a temperature sensor obtained by making a transistor temperature sensor for generating an output current proportional to absolute temperature an integrated circuit. The thermistor is made by sintering a metallic oxide (for example, chrome, cobalt, manganese, nickel, and/or titanium) and may indicate resistance having a large change depending on temperature. For example, the thermistor may be divided into a positive temperature coefficient (PTC) thermistor of which resistance increases as temperature increases and a negative temperature coefficient (NTC) thermistor of which resistance decreases as temperature increases. The RTD indicates a temperature sensor made of pure materials such as metals (for example, platinum, nickel, or copper) and may convert resistance into temperature to measure temperature.

The communication module 220 may include an element corresponding to the wireless communication module 192 as described with reference to FIG. 1. The communication module 220 may support communication with an external device through a first network or a second network. The first network can include, for example, a short-range communication network such as Bluetooth, BLE, WiFi direct, IrDA, and/or ultra-wide band (UWB)). The second network can include, for example, a long-range communication network such as a legacy network (for example, a 3G network and/or a 4G network), a 5G network, or a next-generation communication (for example, new radio (NR)) network, Internet, or a computer network (for example, LAN or WAN)).

The first electronic device 201 may communicate with an external device (for example, the server 108 of FIG. 1) and/or another electronic device 101, 102, or 104 via the communication module 220 through the network. The communication module 220 may transmit data generated by the first electronic device 201 to an external device and receive data transmitted from the external device.

The communication module 220 may include a first communication module 221 for first communication and a second communication module 222 for second communication.

In an embodiment, the first communication module 221 may support establishment of a first wireless communication channel and performance of first communication through the established wireless communication channel. For example, in an embodiment of the disclosure, the first communication module 221 may perform first predetermined communication (for example, Bluetooth, BLE, and/or WiFi communication) with an external device (for example, the electronic device 101 of FIG. 2).

In an embodiment, the second communication module 222 may support establishment of a second wireless communication channel and performance of second communication through the established wireless communication channel. For example, in an embodiment of the disclosure, the second communication module 222 may perform second predetermined communication (for example, UWB) with an external device (for example, the second electronic device 101).

Certain embodiments are not limited thereto, and it may be understood that the first communication module 221 and the second communication module 222 indicate various types of communication modules for performing various types of different communication.

The output module 230 may include elements corresponding to the sound output module 155, the haptic module 179, and/or the display module 160 as described with reference to FIG. 1. The output module 230 may output various pieces of data (for example, a sound signal, mechanical stimulation, and/or an image signal) generated by the first electronic device 201 to the outside of the first electronic device 201 through an output module corresponding thereto on the basis of a type of the first electronic device 201 (for example, a watch type, a ring type, or an in-ear type). In an embodiment of the disclosure, the output module 230 may visually, acoustically, and/or tactually provide various pieces of information related to temperature of the user under the control of the processor 250.

The memory 240 may include an element corresponding to the memory 130 as described with reference to FIG. 1. The memory 240 may store various pieces of data used by the first electronic device 201. The data may include, for example, input data or output data for an application (for example, the program 140 of FIG. 1) and commands related thereto.

The memory 240 may include applications related to the execution of a first function (or operation) of measuring and monitoring (or managing) body temperature of the user. The first function can be performed by the processor 250. The memory 240 may also include applications related to a second function (or operation) of making a connection with an external device (for example, the second electronic device 101) and exchanging data. For example, body temperature measurement may be performed by a health management (for example, body temperature measurement) application, and communication with the external device may be performed by a communication application. The application may be stored in the memory 240 as software (for example, the program 140 of FIG. 1) and may be executed by the processor 250.

The first function may be performed on the basis of estimation of core body temperature, determination of basal body temperature, and/or definition of an environment variable. The second function may perform at least one communication with the external device. The communication may be through the first predetermined communication or both the first predetermined communication and the second predetermined communication. Measurement of body temperature of the user by the first electronic device 201 or the connection with the external device will be described with reference to drawings below. The memory 240 may store various data related to the operation of functions of the first electronic device 201. The data may include reference information 241, correction information 243, sensing data 245, and/or connection information 247.

In an embodiment, the reference information 241 may include at least one reference value (or initial configuration value) for determining whether body temperature of the user is not normal.

In an embodiment, the correction information 243 may include a correction value for estimating core body temperature. The correction information 243 may include a predefined body temperature correction value for each body part according to the type of the first electronic device 201.

In an embodiment, the sensing data 245 may include sensing data acquired from the body thermometer 211.

In an embodiment, the connection information 247 may include information on predetermined communication used when body temperature information is provided to the external device (for example, the second electronic device 101) and information on the connection therefor.

The memory 240 may store at least one module for processing a body temperature measurement function that can be performed by the processor 250. For example, the memory 240 may include at least some of an event detection module 251, a correction module 253, and/or an event processing module 255 of the processor 250 in the form of software (or in the form of instructions).

The processor 250 may control a function (or operation) of determining a reference (for example, the reference information 241) for determining body temperature of the user and measuring body temperature of the user on the basis of the determined reference. When measuring body temperature of the user, the processor 250 may control a function (or operation) of estimating core body temperature obtained by correcting the measured temperature on the basis of a predetermined correction value and calculating actual body temperature of the user. Estimation of the core body temperature may include an operation of correcting and/or complementing, for example, primarily measured body temperature by various pieces of surrounding information or additional information.

The processor 250 may measure body temperature of the user through the sensor module 210 (for example, the body thermometer 211). The measured body temperature can be the temperature that is measured based on directed contact of a body part with the electronic device 201. Based on the measured temperature, the processor 250 may estimate a core body temperature of the user. A core body temperature can be the temperature of an internal organ of the user. The processor 250 may determine body temperature of the user on the basis of core body temperature. The processor 120 may control an operation (or processing) related to provision of body temperature of the user on the basis of the determined body temperature. The processor 250 may detect a predetermined event related to provision of body temperature and control an operation (or processing) related to provision of body temperature information to the external device (for example, the second electronic device 101) through the communication module 220.

The processor 250 may include at least one module for processing a body temperature measurement function. For example, the processor 250 may include the event detection module 251, the correction module 253, and/or the event processing module 255.

The event detection module 251 may detect a predetermined event related to measurement and/or provision of body temperature information on the basis of detection of abnormality of body temperature of the user or detection of a request from the external device (for example, the second electronic device 101). Detection of the event (or trigger) related to measurement of body temperature of the user according to an embodiment is described with reference to drawings below.

The correction module 253 may estimate core body temperature by correcting the measured body temperature of the user. The body temperature may be corrected on the basis of a predefined body temperature correction value for each body part according to the type of the first electronic device 201. In an embodiment, a body temperature correction value for body temperature correction may be differently configured according to the type of the first electronic device 201. The operation of estimating core body temperature may include an operation of correcting and/or complementing, for example, primarily measured body temperature by various surrounding information or additional information.

The event processing module 255 may provide measured body temperature information to the external device (for example, the second electronic device 101) based on detection of the predetermined event. The event processing module 255 may provide body temperature information to the external device through the first communication module 221 or the second communication module 222. In an embodiment, the first communication module 221 may use Bluetooth, BLE, and/or WiFi communication. In an embodiment, the second communication module 222 may use UWB and/or IrDA communication.

At least some of the event detection module 251, the correction module 253, and/or the event processing module 255 may be included in the processor 250 as hardware modules (for example, circuitry), and/or may be implemented as software including one or more instructions that can be executed by the processor 250. For example, operations performed by the processor 250 may be stored in the memory 240 and may be performed by instructions executed by the processor 250 when performed.

The processor 250 according to certain embodiments may control various operations related to the general function of the first electronic device 201 as well as the above functions. For example, the processor 250 may communication with the external device to transmit and receive data and control the output of the transmitted and received data in a predetermined scheme through a predetermined output module. In another example, the processor 250 may receive input signals corresponding to various touch event or proximity event inputs supported by a touch-based or proximity-based input interface and control the operation of functions according thereto.

The first electronic device 201 is not limited to the elements illustrated in FIG. 2, and at least one element may be omitted or added. According to an embodiment, the first electronic device 201 may include a voice recognition module (not shown). For example, the voice recognition module (not shown) may indicate an embedded ASR (eASR) module and/or an embedded NLU (eNLU).

Certain embodiments of the disclosure may be implemented in a recording medium, which can be read through a computer or a device similar thereto, by using software, hardware, or a combination thereof. In certain embodiments, the recording medium may include a computer-readable recording medium recording a program for performing an operation of measuring body temperature of the user through the sensor module 210, an operation of estimating core body temperature of the user on the basis of the measured body temperature, an operation of determining the body temperature of the user on the basis of the core body temperature, and an operation of providing body temperature information of the user on the basis of the determined body temperature.

Electronic Device of the Person Requesting Measuring

FIG. 3 schematically illustrates an example of a block diagram of the second electronic device according to an embodiment of the disclosure. The second electronic device 101 can be use to make a request to the first electronic device 201 to measure the body temperature of another user.

In the disclosure, the second electronic device 101 according to an embodiment may indicate an electronic device of a person to measure (for example, a parent and/or a protector) who desires to measure body temperature of a person whose body temperature is to be measured. The second electronic device 101 may be possessed by the person to measure, and may be a device including a display module (for example, the display module 160 of FIG. 1) which can be monitored, for example, a smartphone, a tablet PC, or a laptop computer, as an electronic device capable acquiring body temperature information of the person to be measured from an external device through communication with the external device (for example, the first electronic device 201) worn on a body part of the person to measure.

Referring to FIG. 3, the second electronic device 101 (for example, the smartphone) according to an embodiment may include a communication module 310, the display module 160, the memory 130, and/or the processor 120.

The communication module 310 may include an element corresponding to the wireless communication module 192 as described with reference to FIG. 1. The communication module 220 may support communication with an external device through a first network (for example, a short-range communication network such as Bluetooth, BLE, WiFi direct, IrDA, and/or ultra-wide band (UWB)) or a second network (for example, a long-range communication network such as a legacy network (for example, a 3G network and/or a 4G network), a 5G network, or a next-generation communication (for example, new radio (NR)) network, Internet, or a computer network (for example, LAN or WAN)).

The second electronic device 101 may communicate with an external device (for example, the server 108 of FIG. 1) and/or another electronic device 201, 102, or 104 via the communication module 310 through the network. The communication module 310 may transmit data generated by the second electronic device 101 to an external device and receive data transmitted from the external device.

The communication module 310 may include a first communication module 311 for first communication and a second communication module 312 for second communication.

In an embodiment, the first communication module 311 may support establishment of a first wireless communication channel and performance of first communication through the established wireless communication channel. For example, in an embodiment of the disclosure, the first communication module 311 may perform first predetermined communication (for example, Bluetooth, BLE, and/or WiFi communication) with the external device (for example, the first electronic device 201).

In an embodiment, the second communication module 312 may support establishment of a second wireless communication channel and performance of second communication through the established wireless communication channel. For example, in an embodiment of the disclosure, the second communication module 312 may perform second predetermined communication (for example, UWB) with the external device (for example, the first electronic device 201).

Certain embodiments are not limited thereto, and it may be understood that the first communication module 311 and the second communication module 312 indicate different types of communication modules for performing different types of communication.

The display module 160 may include an element corresponding to the display module 160 as described with reference to FIG. 1. The display module 160 may visually provide information to the outside (for example, the user) of the second electronic device 101. The display module 160 may include a touch detection circuit (or a touch sensor) (not shown), a pressure sensor capable of measuring the intensity of a touch, and/or a touch panel (for example, a digitizer) capable of detecting a stylus pen in a magnetic type.

The display module 160 may detect a touch input and/or a hovering input (or a proximity input) b measuring a change in a signal (for example, voltage, quantity of light, resistance, electromagnetic signal, and/or quantity of electric charge) for a specific location of the display module 160 on the basis of the touch sensing circuit, the pressure sensor, and/or the touch panel. The display module 160 may include a liquid crystal display (LCD), an organic light emitted diode (OLED), and an active matrix organic light emitted diode (AMOLED). The display module 160 may include a flexible display.

Under the control of the processor 120, the display module 160 may visually provide various information (for example, a user interface) related to a reference configuration for measurement of body temperature of the person to be measured, various information (for example, a user interface) related to performance of measurement of body temperature of the person to be measured, and/or temperature information measured by and received from an external device.

The memory 130 may include an element corresponding to the memory 130 as described with reference to FIG. 1. According to an embodiment, the memory 130 may store various pieces of data used by the second electronic device 101. The data may include, for example, input data or output data for an application (for example, the program 140 of FIG. 1) and commands related thereto.

The memory 130 may include applications related to the execution of a first function (or operation) of configuring a reference for measurement of body temperature of the person to be measured and managing and/or collecting body temperature information of the person to be measured. The memory 130 may also include applications related toa second function (or operation) of making a connection with an external device (for example, the first electronic device 201) and transmitting and receiving data.

For example, body temperature management of the person to be measured may be performed by a health management (for example, body temperature management) application, and communication with the external device may be performed by a communication application. The application may be stored in the memory 130 as software (for example, the program 140 of FIG. 1) and may be executed by the processor 120.

The first function may be performed on the basis of detection of an intention of the user (for example, the person to measure) or detection of the generation of an event (or trigger) from the external device (for example, the first electronic device 201). The second function may perform at least one communication with the external device through the first predetermined communication or both the first predetermined communication and the second predetermined communication. Collection of body temperature information of the person to be measured by the second electronic device 101 or the connection with the external device will be described with reference to drawings below. The memory 130 may store data related to the operation of functions of the second electronic device 101. The data may include connection information 331 and/or biometric information 333 (for example, body temperature for each user).

In an embodiment, the connection information 331 may include information on predetermined communication used for body temperature information is acquired from the external device (for example, the first electronic device 201) and the connection thereof.

In an embodiment, the biometric information 333 may include various pieces of information (for example, heartbeat information, body temperature information, and/or lifespan state information) which can be measured from a user's body.

The memory 130 may store at least one module for processing a body temperature management function which can be performed by the processor 120. For example, the memory 130 may include at least some of an event detection module 351, an event processing module 353, and/or a connection module 355 in the form of software (or in the form of instructions).

The processor 120 may designate an external device for body temperature measurement through predetermined communication and control a function (or operation) for acquiring body temperature information from the designated external device and providing the same.

The processor 120 may search for at least one neighboring external device (for example, the first electronic device 201 of the person to be measured) on the basis of a user and provide a predetermined list on the basis of at least one found external device. The processor 120 may detect a predetermined event for acquiring body temperature information measured by the external device and acquire body temperature information from the predetermined external device through the communication module 310. The processor 120 may provide body temperature information through the display module 160 on the basis of a predetermined user interface.

The processor 120 may search for at least one external device in a predetermined space within a predetermined range through first predetermined communication for designating the range and determine an external device of a target whose body temperature is to be measured in a predetermined direction and/or distance among at least one external device through second predetermined communication for designating the target whose body temperature is to be measured.

The processor 120 may include at least one module for processing a body temperature management function. For example, the processor 120 may include the event detection module 351, the event processing module 353, and/or the connection module 355.

The event detection module 351 may detect a predetermined event (or trigger) for acquiring body temperature information measured by an external device (for example, the first electronic device 201). In an embodiment, the predetermined event may include reception of a notification for detection of an abnormality signal from the external device and/or a user input related to a body temperature measurement intention. Detection of the event (or trigger) related to the event (or trigger) related to measurement of body temperature of the person to be measured according to an embodiment is described with reference to drawings below.

The event processing module 353 may acquire body temperature information from an external device (for example, the first electronic device 201) through the predetermined communication module 310 on the basis of detection of a predetermined event. The event processing module 353 may transmit a body temperature information request to the external device through the first predetermined communication module (for example, the first communication module 311 of FIG. 3) for first predetermined communication or the second predetermined communication module (for example, the second communication module 312 of FIG. 3) for second predetermined communication different from the first predetermined communication. In an embodiment, the first predetermined communication may include Bluetooth, BLE, and/or WiFi communication. In an embodiment, the second predetermined communication may include UWB and/or IrDA communication.

The connection module 355 may process the operation of the connection with the external device, associated with recognition of at least one external device in a predetermined space within a predetermined range through first predetermined communication for range designation and designation of an external device of a target whose body temperature is to be measured in a predetermined direction and/or distance among at least one external device through second predetermined communication for designating the target whose body temperature is to be measured.

According to an embodiment, at least some of the event detection module 351, the event processing module 353, and/or the connection module 355 may be included in the processor 120 as hardware modules (for example, circuits) or may be implemented as software including one or more instructions which can be executed by the processor 120. For example, operations performed by the processor 120 may be stored in the memory 130 and may be performed by instructions executed by the processor 120 when performed.

According to certain embodiments, the processor 120 may control various operations related to the general function of the electronic device 101 as well as the above function. For example, when a predetermined application is executed, the processor 120 may control the operation and screen display thereof. In another example, the processor 120 may perform control to transmit and receive data through communication with an external device and display the transmitted and received data through the display module 160. In another example, the processor 120 may receive input signals corresponding to various touch event or proximity event inputs supported by a touch-based or proximity-based input interface and control the operation of functions according thereto.

According to certain embodiments, the second electronic device 101 is not limited to the elements illustrated in FIG. 3, and at least one element may be omitted or added. The second electronic device 101 may include a voice recognition module (not shown). For example, the voice recognition module (not shown) may indicate an eASR module and/or an eNLU.

The certain embodiments of the disclosure may be implemented in a recording medium, which can be read through a computer or a device similar thereto, by using software, hardware, or a combination thereof. In certain embodiments, the recording medium may include a computer-readable recording medium recording a program for performing an operation of searching for at least one neighboring external device on the basis of a user input, an operation of providing a predetermined list on the basis of at least one found external device, an operation of detecting a predetermined event for acquiring body temperature information measured by the external device, an operation of acquiring body temperature information from the predetermined external device through a communication module, and an operation of providing the body temperature information through the display module 160 on the basis of a predetermined user interface.

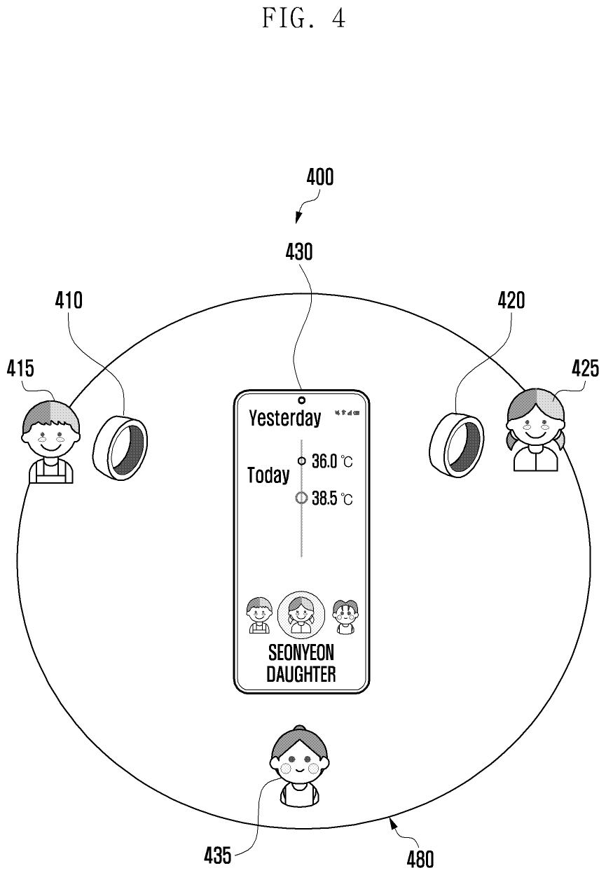

FIG. 4 illustrates an example of the operation for measuring body temperature of the person associated with the first electronic device in a system that include the first electronic device and a second electronic device according to an embodiment of the disclosure.

A person 435 can request the body temperature of either person 415 or person 425. Person 435 can request the body temperature of either person 415 or person 425 by making a request on electronic device 430. The electronic device 430 can detect electronic devices 410 and 420 that are proximate to electronic device 430. User 435 can then identify which one of persons 415 or 425 to take the temperature of. The electronic device 430 sends a request to the electronic device 410 or 420 associated with the selected person. In response, electronic device 410 or 420, as the case may be, take the temperature of the user and provides the information to electronic device 430.

According to an embodiment, FIG. 4 illustrates an example of a body temperature measurement system 400 including a person to be measured (for example, electronic devices 410 and 420 (for example, the first electronic device 201) of a first person 415 to be measured and a second person 425 to be measured and an electronic device 430 (for example, the second electronic device 101 of a person to measure (for example, a person 435 to measure)).

For example, FIG. 4 shows an example of the system 400 related to an operation of measuring body temperature of the person to be measured on the basis of a mutual operation between the first electronic device 201 and the second electronic device 101 and sharing the body temperature of the person to be measured in the state in which the first electronic device 201 and the second electronic device 101 coexist in a predetermined space (or limited space).

The electronic devices 410 and 420 may associated with people 415 and 425 whose temperature may be measured (for example, protected people or children). For example, the electronic devices 410 and 420 may indicate the first electronic device 201 including a sensor module (for example, the sensor module 210 of FIG. 2) capable of measuring body temperature of the people 415 and 425 to be measured. The first electronic device 201 may include a wearable device such as a device in a watch type, a ring type, and/or an in-ear type (for example, earbud).

The electronic device 430 may indicate an electronic device of the person 435 to measure (for example, a protector or a parent) who desires to identify body temperature of the people 415 and 425 to be measured. For example, the electronic device 430 may indicate the second electronic device 101 capable of collecting body temperature information of the people 415 and 425 to be measured from the electronic devices 410 and 420 of the people 415 and 425 to be measured and manage the collected body temperature information. The second electronic device 101 may include a device such as a smartphone, a tablet PC, and/or a laptop computer.

It is noted that the foregoing is illustrative and not limiting. For example, person 435 may also be associated with an electronic device similar to electronic devices 410 and 420, and the electronic device may identify the person, and collect their body temperature.

Measurement of body temperature of the user (for example, the person 415 or 425 to be measured) may include an on-demand type in which the electronic device 410 or 420 capable of measuring body temperature is driven to measure body temperature when needed and an unconscious type in which body temperature can be measured in an unconscious state of the user. In an embodiment, an example in which body temperature of the people 415 and 425 to be measured is measured and provided in the unconscious type in which the electronic devices 410 and 420 (for example, the first electronic device 201) of the people 415 and 425 to be measured can measure body temperature of the people 415 and 425 to be measured even in the unconscious state of the people 415 and 425 to be measured is described.

The electronic devices 410 and 420 (for example, the first electronic device 201) of the people 415 and 425 to e measured may regularly measure body temperature of the people 415 and 425 to be measured in the unconscious type and store and manage the regularly measured body temperature information. The electronic devices 410 and 420 of the people to be measured may provide body temperature information to the people 415 and 425 to be measured on the basis of requests from the people 415 and 425 to be measured. The electronic devices 410 and 420 of the people to be measured may provide body temperature information to the people 415 and 425 to be measured on the basis of a predetermined trigger (for example, larger than a reference by a predetermined value or more and/or detection of abnormality). According to some embodiments, the electronic devices 410 and 420 of the people to be measured may transmit body temperature information of the people 415 and 425 to be measured according to a request from the outside (for example, the electronic device 430 of the person to measure (for example, the first electronic device 201)) to provide the same to an external user (for example, the person 435 to measure).

In embodiments of the disclosure, as illustrated in FIG. 4, it is possible to improve the usability and accuracy by supporting more convenient and accurate measurement of body temperature of the people 415 and 425 by the person 435, when all are in a predetermined space. The disclosure shows an example of measuring body temperature in a short-distance in which the people 415 and 425 to be measured come into sight of the person 435 rather than remotely measuring the body temperature. This prevents misrecognition of measurement of body temperature of the people 415 and 425 to be measured.

The operation for measuring body temperature of the people 415 and 425 by the person 435 in a predetermined space 480 (or limited space) (for example, an environment within home) may be within a predetermined range (for example, within a radius of about 10 m) through first predetermined communication (for example, Bluetooth, BLE, and/or WiFi communication). For example, the first predetermined communication is communication for range designation and may include Bluetooth, BLE, and/or WiFi communication capable of establishing a first network (for example, Bluetooth, BLE, and/or WiFi networks) for the predetermined range.

The first predetermined communication (for example, BLE) may indicate communication for initially identifying the existence of the electronic devices 410 and 420 (for example, the first electronic device 201) of the people 415 and 425 and the electronic device 430 (for example, the second electronic device 101) of the person 435 in the same space 480.

The disclosure describes the operation within the predetermined space 480, but is not limited thereto. For example, the corresponding operation may be provided when a distance between the person to measure (for example, the person 435 to measure) and the person to be measured (for example, the first person 415 to be measured and/or the second person 425 to be measured) is beyond a predetermined range (or distance). When the person 415 or 425 to be measured (for example, the protected person) or the person 435 to measure (for example, the protector or the parent) in the predetermined space 480 (for example, within home) escapes the predetermined space 480, the electronic device 430 (for example, the second electronic device 101) of the person 435 to measure may deactivate and display an icon object (for example, a family icon object) related to the person 415 or 425 to be measured on a screen (for example, the screen of the electronic device 430 of FIG. 4) performing the body temperature measurement function and provide location information related to the location of the corresponding person to be measured.

When the person 435 to measure attempts measurement of body temperature of the person 415 or 425 to be measured, if the person to be measured is not located within a measurable distance (for example, a predetermined range), the electronic device 430 (for example, the second electronic device 101) of the person 435 to measure may activate a remind function. For example, when a relatively distance between the person 435 to measure and the person 415 or 425 to be measured is within a predetermined range (for example, coexist in the predetermined space 480) or the person 435 to measure and the person 415 or 425 to be measured are located within a geofence such as a predetermined place (for example, the predetermined space 480 such as home and/or a car), the electronic device 430 (for example, the second electronic device 101) may provide a notification to allow the person 435 to measure to measure again body temperature of the person 415 or 425 to be measured.

As illustrated in FIG. 4, the person 435 to measure may monitor several people 415 and 425 to be measured. In the disclosure, in order to acquire body temperature of a predetermined target among the several people 415 and 425 to be measured, the person 435 to measure may use second predetermined communication (for example, UWB and/or IrDA communication) for determining the relative location (or direction and distance) of the person 435 to measure and the person 415 or 425 to be measured. For example, the second predetermined communication is communication for designating the target whose body temperature is measured and may include UWB and/or IrDA communication.

The electronic device 410 or 420 (for example, the first electronic device 201) of the person 415 or 425 to be measured and the electronic device 430 (for example, the second electronic device 101) of the person 435 to measure may recognize the existence in a predetermined space through first predetermined communication. The first electronic device 201 and the second electronic device 101 may determine (or select) one electronic device 410 or 420 among the electronic devices 410 and 420 of the people 415 and 425 to be measured through second predetermined communication and designate the person to be measured, whose body temperature is to be measured. The first electronic device 201 and the second electronic device 101 may be connected to each other or recognized through the first predetermined communication (for example, BLE), and the second predetermined communication may be turned on when a predetermined condition is triggered (for example, detection of initiation of the body temperature measurement operation) in an turned-off state in consideration of power consumption.

Referring to the example of FIG. 4, the electronic device 430 of the person 435 to measure and the electronic device 430 of the person 415 or 425 to be measured may be connected through the first predetermined communication (for example, BLE) in the predetermined space 480. The electronic device 430 of the person 435 to measure may determine an electronic device of a person to be measured, located within a predetermined distance and/or in a predetermined direction among the electronic devices 410 and 420 of the plurality of people 415 and 425 to be measured, existing in the predetermined space 480 through the second predetermined communication and acquire body temperature information of the person to be measured from the electronic device of the corresponding person to be measured. The electronic device of the corresponding person to be measured may detect initiation of the operation of measuring the body temperature of the person to be measured through the second predetermined communication with the electronic device 430 of the person 435 to measure and provide body temperature information of the person to be measured to the electronic device 430 of the person 435 to measure through the first predetermined communication and/or the second predetermined communication.

As illustrated in FIG. 4, the electronic device 430 (for example, the second electronic device 101) of the person 435 to measure and the electronic devices 410 and 420 (for example, the first electronic device 201) of the people 415 and 425 to measure may measure and provide body temperature on the basis of a predetermined (or limited) distance and/or direction. It is assumed that the person 435 to measure views the person 415 or 425 and/or pays attention to the same. The disclosure may provide more accurate and intuitive body temperature measurement by providing conditions of enabling monitoring of various factors (for example, food intake, surrounding temperature, and/or fever due to diseases) influencing measurement of body temperature of the person 415 or 425 to be measured by the person 435 to measure.

It is possible to estimate the conditions of various factors influencing measurement of body temperature of the person 415 or 425 to be measured through recognition of the condition (for example, behavior or surrounding object) of the person 415 or 425 to be measured through a camera module (for example, the camera module 180 of FIG. 1) included in the electronic device 430 (for example, the second electronic device 101) of the person 435 to measure. The electronic device 430 of the person 435 to measure may receive the factor (for example, surrounding temperature information) influencing body temperature of the person 415 or 425 to be measured in an IoT environment and determine whether the factor influences the body temperature of the person 415 or 425 to be measured.

An electronic device (for example, the first electronic device 201 of FIG. 2) according to certain embodiments of the disclosure includes a sensor module (for example, the sensor module 210 of FIG. 2), a communication module (for example the communication module 220 of FIG. 2), and a processor (for example, the processor 250 of FIG. 2) operatively connected to the sensor module 210 and the communication module 220, wherein the processor 250 may be configured to measure body temperature of a user through the sensor module 210, estimate core body temperature of the user, based on the measured body temperature, determine the body temperature of the user, based on the core body temperature, and provide body temperature information of the user, based on the determined body temperature, and the measured body temperature may include shell temperature of a body part of a body of the user on which the electronic device is worn and the core body temperature may include core temperature of an internal organ of the body of the user.

The processor 250 may be configured to estimate the core body temperature by correcting the measured body temperature on the basis of a predefined body temperature correction value for each body part according to a type of the electronic device 201, and the body temperature correction value for correcting the body temperature is differently configured according to the type of the electronic device 201.

The processor 250 may be configured to detect a predetermined event related to provision of body temperature information and provide the body temperature information to an external device (for example, the second electronic device 101 of FIG. 3) through the communication module 220.

The processor 250 may be configured to provide body temperature information to the external device (for example, the second electronic device 101 of FIG. 3) through a first predetermined communication module (for example, the first communication module 221 of FIG. 2) for first predetermined communication or a second predetermined communication module (for example, the second communication module 222 of FIG. 2) for second predetermined communication different from the first predetermined communication, the first predetermined communication may include Bluetooth, BLE, and/or WiFi communication, and the second predetermined communication may include UWB and/or IrDA communication.

The processor 250 may be configured to determine the predetermined event related to provision of body temperature information on the basis of detection of abnormality of body temperature of the user or detection of a request from the external device (for example, the second electronic device 101 of FIG. 3).

The electronic device 201 may include wearable devices such as watch type, ring type, or in-ear type devices.

An electronic device (for example, the second electronic device 101 of FIG. 3) according to certain embodiments of the disclosure may include a communication module (for example, the communication module 310 of FIG. 3), a display module (for example, the display module 160 of FIG. 1 or FIG. 3), and a processor (for example, the processor 120 of FIG. 1 or FIG. 3) operatively connected to the communication module 310 and the display module 160, wherein the processor 120 may be configured to search for at least one neighboring external device (for example, the first electronic device 201 of FIG. 2) on the basis of a user input, provide a predetermined list on the basis of at least one found external device, detect a predetermined event for acquiring body temperature information measured by the external device, acquire body temperature information from a predetermined external device through the communication module 310, and provide the body temperature information through the display module 160 on the basis of a predetermined user interface.

The user input may include an input for executing a predetermined application for managing body temperature, and the processor 120 may be configured to search for neighboring external devices through first predetermined communication on the basis of the user input.

The processor 120 may be configured to transmit a body temperature information request to the external device through a first predetermined communication module (for example, the first communication module 311 of FIG. 3) for first predetermined communication or a second predetermined communication module (for example, the second communication module 312 of FIG. 3) for second predetermined communication different from the first predetermined communication, the first predetermined communication may include Bluetooth, BLE, and/or WiFi communication, and the second predetermined communication may include UWB and/or IrDA communication.

The processor 120 may be configured to search for the at least one external device in a predetermined space within a predetermined range through the first predetermined communication for designating the range and determine an external device of a target whose body temperature is to be measured in a predetermined direction and/or distance among the at least one external device through the second predetermined communication for designating the target whose body temperature is to be measured.

The processor 120 may be configured to recognize at least one external device existing in a predetermined space through the first predetermined communication, designate one external device of the at least one external device as an external device for measuring body temperature through the second predetermined communication, and acquire the body temperature information from the designated external device through the second predetermined communication.

Hereinafter, a method of operating the first electronic device 201 and the second electronic device 101 according to certain embodiments is described in detail.

Operations performed by the first electronic device 201 may be performed by a processor (for example, the processor 250 of FIG. 2) including at least one processing circuit (circuitry) of the first electronic device 201. According to an embodiment, operations performed by the first electronic device 201 may be performed by instructions that are stored in a memory (for example, the memory 240 of FIG. 2) and cause the processor 250 to operate when executed.

Operations performed by the second electronic device 101 may be performed by a processor (for example, the processor 120 of FIG. 1 or FIG. 3) including at least one processing circuit of the second electronic device 101. According to an embodiment, operations performed by the second electronic device 101 may be performed by instructions that are stored in a memory (for example, the memory 130 of FIG. 1 or FIG. 3) and cause the processor 120 to operate when executed.

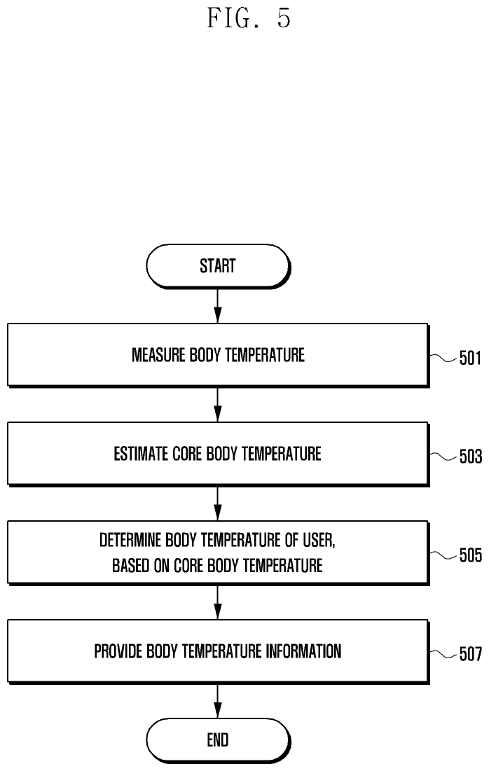

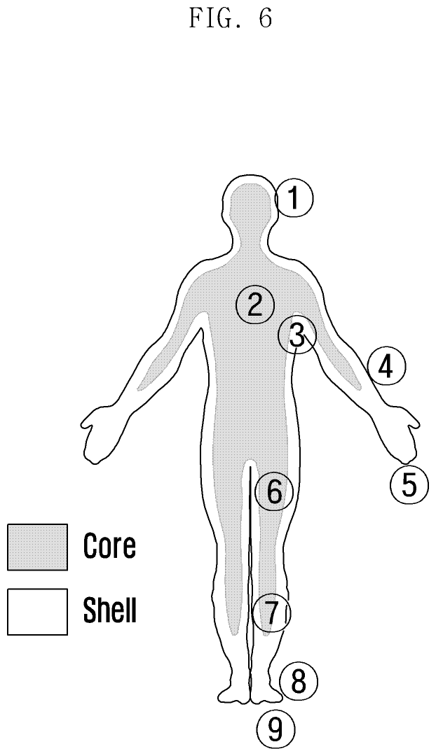

FIGS. 5 and 6 describe how electronic device 410, 420 provide body temperature information.

FIG. 5 is a flowchart illustrating a method of operating an electronic device according to an embodiment of the disclosure.

FIG. 6 illustrates an example of distribution of core temperature and shell temperature of the body.

According to an embodiment, FIG. 5 shows an example of the operation in which the first electronic device 201 (for example, the electronic device 410 or 420 of the person 415 or 425 to be measured of FIG. 4) measures body temperature of a user (for example, the person to be measured).

Referring to FIG. 5, in operation 501, the processor 250 of the first electronic device 201 may measure body temperature of the user. The first electronic device 201 may periodically measure body temperature of the user through the sensor module 210 (for example, the body thermometer 211), and the processor 250 may collect sensor data related to body temperature from the sensor module 210. The processor 250 may collect sensor data (for example, measured body temperature information) measured by the sensor module 210 on the basis of a predetermined event (or trigger) (for example, detection of a predetermined time, detection of a user request, detection of an abnormality signal, and/or detection of reception f an external request). The measured body temperature may be the body temperature that is measured by electronic device 410, 420 by direct exposure the surface of the user's body.

In operation 503, the processor 250 may estimate core body temperature. The operation of estimating core body temperature may include an operation of correcting and/or complementing, for example, primarily measured body temperature by various surrounding information or additional information. The processor 120 may estimate core body temperature of the user on the basis of the measured body temperature. The processor 250 may estimate core body temperature by correcting the measured body temperature on the basis of a predefined body temperature correction value for each body part according to the type of the first electronic device 201. The measured body temperature may indicate shell temperature of the body part (for example, the end of the body on which the first electronic device 201 is worn in the user's body). The core body temperature may indicate temperature (for example, core temperature) of internal organs of the body and may be distinguished from temperature of the end of the body. An example thereof is described in FIG. 6 and [Table 1]. It is noted that electronic device 410, 420 are primarily worn about a particular part of the body, such as wrist, finger, ankle, and head. The temperature of the body can be different at different parts of the body. For example, the feet are considerably colder than the rest of the body. Accordingly, a measured body temperature may be different from the internal body temperature, or core temperature.

FIG. 6 shows comparison between core temperature and shell temperature of the body. Table 1 may indicate distribution of temperature for each body part at predetermined temperature (for example, current surrounding temperature). In FIG. 6, the hatching part may indicate core temperature and the blank part may indicate shell temperature. In FIG. 6, reference numerals \textcircled{1} to \textcircled{2} indicate body parts in [Table 1].

TABLE 1

| Body part | Average body temperature | Correction |
|---|---|---|
| Scalp | About 34.8 C./94.64 F. | 2.2 C./3.96 F. |
| Chest | About 34.5 C./94.1 F. | 2.5 C./4.5 F. |
| Axilla | About 36.4 C./97.52 F. | 0.6 C./1.08 F. |
| Arm | About 33.5 C./92.3 F. | 3.5 C./6.3 F. |
| Finger | About 33.2 C./91.76 F. | 3.8 C./6.84 F. |
| Thigh | About 33.4 C./92.12 F. | 3.6 C./6.48 F. |
| Leg | About 30.1 C./86.18 F. | 6.9 C./12.42 F. |
| Foot | About 29.7 C./84.46 F. | 7.3 C./14.14 F. |
| Toe | About 29.1 C./84.38 F. | 7.9 C./14.22 F. |

In general, core temperature of the human body may be about 37 degrees (° C.)/98.6 F and body temperature on the surface of skin may be averagely about 34 to 35 degrees. According to an embodiment, body temperature may be meaningful when it is used as a medically absolute value, and accordingly, a body part to be measured by a device for measuring body temperature may be predetermined. The first electronic device 201 according to the disclosure may be a wearable device such as a device in a watch type, a ring type, and/or an in-ear type (for example, earbud), and a body part on which the device is worn may vary depending on the corresponding type.

According to certain embodiments, the processor 250 may correct the measured body temperature to be relatively closer to the absolute value of body temperature. The correction may be on the basis of the type of the first electronic device 201.

In an embodiment, the correction value for correcting body temperature may vary depending on the type of the first electronic device 201. For example, a correction value can be added to the measured temperature. The correction amount can the amount shown in the correction column of Table 1. The amounts in the correction column of Table 1, are the difference between the average body temperature at the indicated location, and the empirically known average body temperature of 37 C/98.6 F. For example, when a body part on which the first electronic device 201 is worn becomes different, the processor 120 may differently configure the correction value.

When the first electronic device 201 is a wearable device in an in-ear type (for example, earbud) and body temperature is measured by the body thermometer 211 thereof, the body temperature can be measured at an ear closest to the core, and thus a degree of the correction may be relatively low. According to another embodiment, when the first electronic device 201 is a wearable device in a ring type (for example, a ring type wearable device) and body temperature is measured by the body thermometer 211 thereof, the body temperature can be measured at the end of the body (for example, \textcircled{5} finger in FIG. 6 and [Table 1]) and there may be large temperature difference between users wearing the first electronic device 201, and thus a degree of the correction may be relatively high.

The first electronic device 201 may be predefined for a basic correction value according to the type of the first electronic device 201. According to an embodiment, referring to FIG. 6 and [Table 1], it may be noted that there is difference in predetermined temperature (for example, current surrounding temperature) from a reference of averagely about 28 to 30 degrees for each body part. For example, scalp temperature (for example, \textcircled{1} scalp temperature of about 34.8 degrees) has difference of about −1.6 degrees from core body temperature (for example, \textcircled{3} axilla temperature of about 36.4 degrees), arm temperature (for example, \textcircled{4} arm temperature of about 33.5 degrees) has difference of about −2.9 degrees therefrom, and finger temperature (for example, \textcircled{5} finger temperature of about 33.2 degrees) has difference of about −3.2 degrees therefrom. As described above, the first electronic device 201 worn on a part corresponding to the body may measure a different body temperature value according to the type (for example, a watch type, a ring type, or an in-ear type), and may estimate core body temperature by inversely calculating the measured body temperature value in the corresponding type of the first electronic device 201.

A reference value accumulated and updated by the processor 250 may be used for a reference value of the first electronic device 201 for estimating core body temperature. When the person who measure has a measurement value of another part that more accurately approximating the core body temperature of the person to be measured, the measurement value may be used instead of a predetermined correction value configured in advance in the first electronic device 201.

In operation 505, the processor 250 may determine body temperature of the user on the basis of the core body temperature. The processor 250 may determine core body temperature obtained by correcting the measured body temperature on the basis of the predetermined correction value as actual body temperature of the user.

In operation 507, the processor 250 may provide body temperature information of the user on the basis of the determined body temperature. The processor 250 may generate body temperature information on the basis of the determined body temperature. The processor 250 may store, manage, and display the temperature information and/or transmit the same to the outside. The body temperature information may include the determined body temperature value and time information (for example, a time stamp) on a time point at which the body temperature is measured. The body temperature information may include location information related to a place in which the corresponding body temperature is measured and/or device information (for example, type information) of the first electronic device 201 having measured the corresponding body temperature.

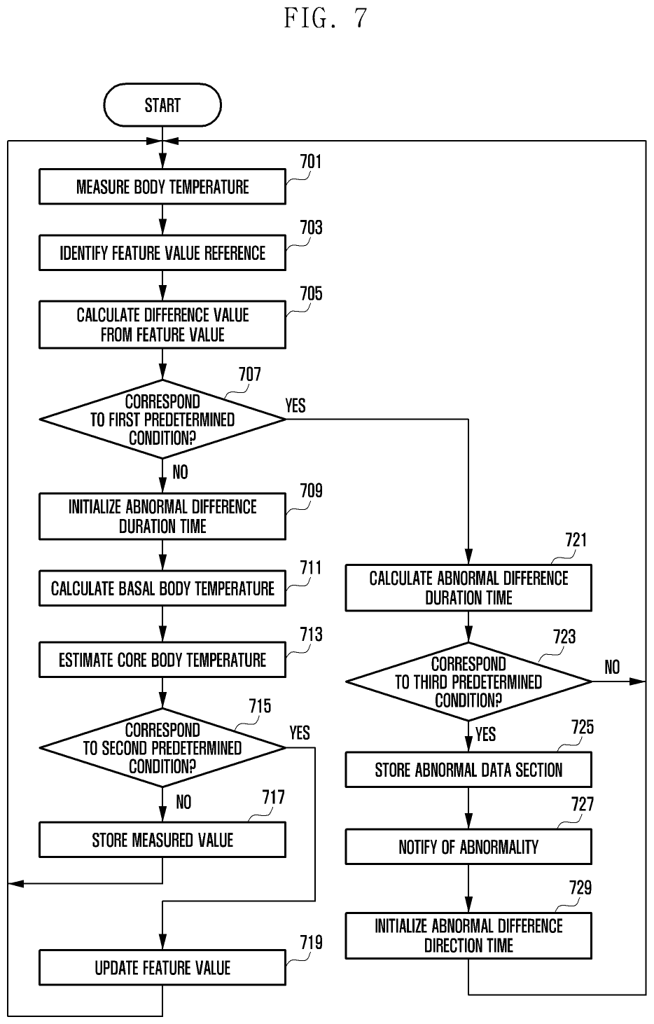

FIG. 7 is a flowchart illustrating a method of operating an electronic device according to an embodiment of the disclosure.

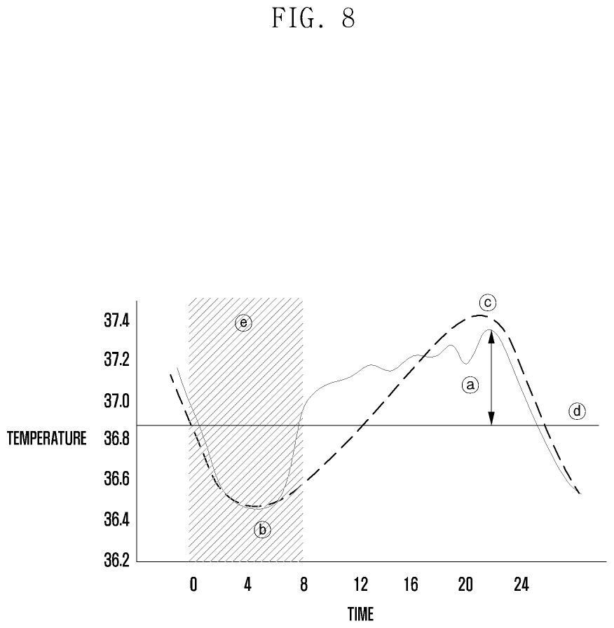

FIG. 8 illustrates a body temperature period graph and examples of feature values according to an embodiment.

According to an embodiment, FIGS. 7 and 8 show an example of the operation in which the first electronic device 201 (for example, the electronic device 410 or 420 of the person 415 or 425 to be measured in FIG. 4) measures body temperature of a user (for example, the person to be measured).

Referring to FIG. 7, in operation 701, the processor 250 of the first electronic device 201 may measure body temperature of the user. The first electronic device 201 may periodically (or according to a predetermined period) measure body temperature of the user through the sensor module 210 (for example, the body thermometer 211), and the processor 250 may collect sensor data related to body temperature from the sensor module 210. When there is no limitation in power consumption of the first electronic device 201, the processor 250 may routinely measure body temperature of the user.

In operation 703, the processor 250 may identify a feature value reference. The feature value may indicate at least one feature for deriving, for example, basal body temperature (BBT) of the user. The Basal Body Temperature is the temperature when the use is fully at rest, such as immediately after waking up in the morning. The basal body temperature may indicate body temperature having no change according to muscular exercise of the user (for example, the person to be measured), food intake, or mental strain and measured during sleeping in which body temperature is stable. The basal body temperature and the feature value are described with reference to FIG. 8. In an embodiment, FIG. 8 shows a body temperature period graph and examples of feature values.

When a predetermined time point arrives in daily periods (for example, about 24 hours), the processor 250 may derive all feature values on the basis of measurement values (for example, sensor data) measured and stored until the predetermined time point. In an embodiment, the predetermined time point may indicate, for example, a reference time point in daily periods, and may include a specific time point before a pre-calculated average sleep onset time. The processor 250 may accumulate feature values derived during the daily periods and update the values to previously calculated feature values as described above. The derived feature value may be a comparison value (for example, a reference value) for calculating difference from a body temperature value to be measured in the next daily periods.

The basal body temperature may indicate, for example, body temperature in the state in which no activity is conducted after the user wakes up after a sufficient sleep, and may be body temperature corresponding to a unique reference that is different for each user. In an embodiment, in order to derive basal body temperature of the user (for example, the person to be measured), the processor 250 may extract various features from the measured body temperature for a predetermined period (for example, daily periods) and manage the same. For example, a circadian rhythm of the user may be a biometric process executed in about 24-hour periods (for example, daily periods), and a biorhythm may be a source of a change in body temperature of the user. Accordingly, the body temperature of the user may also have a regular pattern in about 24-hour periods.

When a graph (for example, FIG. 8) of about 24-hour periods of body temperature is approximated to a cosine function, at least one feature may be identified. For example, as features used for analyzing the relation between body temperature and diseases, acrophase (or a peak time), midline estimating statistic of rhythm (MESOR), and a degree of a change in a height of waves (for example, rhythm curves) in the given period graph (for example, amplitude (for example, an interval between the peak of the waves and the MESOR (or ½ between the peak and the trough))) may be included.

Each feature for daily periods may be changed by influence of the inner parts of the body and an external environment. For example, in the case of an active person, amplitude (for example, the amplitude (\textcircled{a}) in FIG. 8) of the period graph may be larger compared to the case of an inactive person. In another example, in the case of a user lacking sleep (for example, a sleep section (\textcircled{a}) in FIG. 8), amplitude (for example, the amplitude (\textcircled{a}) in FIG. 8) may be reduced due to an increase in the lowest temperature (for example, the trough (\textcircled{b}) in FIG. 8). In another example, in the case of a user who does not wake up at a fixed time or is exposed to light during sleep, the period graph may move and accordingly a time point of the acrophase (for example, the peak (\textcircled{c}) in FIG. 8) may not be constant.

The first electronic device 201 may configure and manage various feature values for daily periods and use the same for calculating basal body temperature. For example, as illustrated in FIG. 8, a value measured during sleep (for example, the sleep section (\textcircled{e}) in FIG. 8) may be used for calculating basal body temperature. The processor 250 may use the lowest value of the trough (\textcircled{b}) generated during a user's sleep (for example, the sleep section \textcircled{e}) in FIG. 8), an intermediate value according to a reference line (\textcircled{d}), and/or a difference value between the lowest value and the intermediate value as feature values.

According to an embodiment, since the user may have many activities in daytime of one day, a change in body temperature may be larger than that in night, and the change in body temperature may be used as the feature value. For example, the processor 250 may accumulate and manage the change in body temperature as the feature value and, accordingly, monitor and detect a change in body temperature beyond a range of the user at ordinary times.

The feature value may be linked and updated with information on an exercise, food intake, and/or a surrounding temperature change that may influence the body temperature of the user during a user's activity. The processor 250 may continuously update the feature value on the basis of the measured body temperature value, and a reliable feature value for body temperature obtained from a measurement part of the user (for example, the person to be measured) may be used as the feature value.

Referring back to FIG. 7, in operation 705, the processor 250 may calculate difference from the feature value. The processor 250 may compare the measured body temperature value on the basis of the feature value and calculate the difference value.

In operation 707, the processor 250 may determine whether the calculated difference value corresponds to a first predetermined condition. The processor 250 may determine whether the difference value is larger than a first predetermined threshold value. In an embodiment, a first predetermined condition (for example, the first predetermined threshold value) may indicate an initial configuration value for determining whether body temperature of the user is abnormal. For example, the first predetermined threshold value may include a reference value by which the processor 250 may determine an abnormal body temperature change range of the user (for example, the person to be measured) and/or a specific range. The first predetermined threshold value (for example, the initial configuration value) according to an embodiment is described with reference to the following drawings.

When the calculated difference value does not correspond to the first predetermined condition (for example, ‘No’ of operation 707), for example, when the calculated difference value is equal to or smaller than the first predetermined threshold value in operation 707, the processor 250 may initialize an abnormal difference duration time in operation 709. According to an embodiment, when the difference value between the measured body temperature value and the feature value is not larger than or equal to a preset reference range while the difference between the measured body temperature value and previously derived feature values is continuously monitored, the processor 120 may initialize an abnormal difference duration time.

In operation 711, the processor 250 may determine (for example, calculate) basal body temperature. According to an embodiment, when the difference value is equal to or smaller than the first predetermined threshold value, the processor 250 may calculate basal body temperature on the basis of the identified feature value. For example, when a value measured during sleep is used as the feature value, the processor 250 may calculate basal body temperature on the basis of the lowest value (for example, the trough \textcircled{b} in FIG. 8) generated during sleep as the feature value. According to an embodiment, since body temperature data used for calculating basal body temperature should be a valid value, the processor 250 may determine a wearing state of the first electronic device 201 and use a value at a time point at which wearing of the first electronic device 201 is identified. The processor 250 may determine the wearing state of the first electronic device 201 through the sensor module 210 (for example, a proximity sensor, a PPG sensor, and/or a motion sensor) of the first electronic device 201.

The processor 250 may determine a sleep time used for calculating basal body temperature through activity recognition of the user in the state in which the first electronic device 201 is worn. For example, in order to determine a sleep state of the user, the processor 250 may identify that the current time zone is a time zone during which people averagely sleep and determine a condition under which a user's movement exists and becomes weak. For example, the user has little movement during sleep on average, but a minute movement may be detected. Subsequently, the processor 250 may measure heartbeat information of the user through the sensor module 210 (for example, a PPG sensor) and analyze the measured heartbeat information in a time/frequency domain so as to determine the sleep state. The processor 250 may determine the sleep state of the user (for example, light, deep, or REM sleep) through the above operation and acquire cycle information.

In operation 713, the processor 250 may estimate core body temperature. The processor 250 may estimate core body temperature of the user on the basis of the measured body temperature as described with reference to FIGS. 5 and 6. The processor 250 may estimate core body temperature by correcting the measured body temperature on the basis of a predefined body temperature correction value for each body part according to the type of the first electronic device 201.

In operation 715, the processor 250 may determine whether a second predetermined condition is satisfied. The processor 250 may complete calculation of basal body temperature and estimation of core body temperature, and determine whether a feature value calculation reference time arrives. The second predetermined condition may indicate a predetermined time point in daily periods. For example, the processor 250 may determine whether any predetermined time point in daily periods arrives.

When the second predetermined condition is not satisfied (for example, ‘No’ of operation 715), for example, when any predetermined time point in daily periods does not arrive in operation 715, the processor 250 may store a measured value (for example, a difference value, basal body temperature, and/or core body temperature) in operation 717. The processor 250 may store the measured value, proceed to operation 701, and perform operations after operation 701.

When the second predetermined condition is satisfied (for example, ‘Yes’ of operation 715), for example, when any predetermined time point in daily periods arrives in operation 715, the processor 250 may update the feature value in operation 719. For example, the processor 250 may update previously calculated feature values on the basis of feature values derived during daily periods and update a reference of the feature value. The processor 250 may update the feature value, proceed to operation 701, and perform operations after operation 701.

When the calculated difference value corresponds to the first predetermined condition (Yes' of operation 707), for example, when the calculated difference value is larger than the first predetermined threshold value in operation 707, the processor 250 may calculate an abnormal difference duration time in operation 721. The processor 250 may calculate a duration time during which the difference between the measured body temperature and the feature value is larger than or equal to a preset reference range while the difference the measured body temperature value and previously derived feature values is continuously monitored.

In operation 723, the processor 250 may determine whether the calculated duration time corresponds to a third predetermined condition. The processor 250 may determine whether the duration time is larger than the second predetermined threshold value. In an embodiment, the third predetermined condition (for example, the second predetermined threshold value) may indicate an initial configuration value for determining whether body temperature of the user is abnormal. For example, the second predetermined threshold value may include a predetermined reference time by which the processor 250 may determine a duration time during which abnormal body temperature of the user (for example, the person to be measured) continues.

When the duration time does not correspond to the third predetermined time (No′ of operation 723), for example, when the calculated duration time is equal to or smaller than the second predetermined threshold value in operation 723, the processor 250 may proceed to operation 707 and perform operations after operation 707.

When the duration time corresponds to the third predetermined condition (for example, ‘Yes’ of operation 723), for example, when the calculated duration time is larger than the second predetermined threshold value in operation 723, the processor 250 may store an abnormal data section in operation 725. According to an embodiment, when the difference between the measured body temperature value and the feature value, which is larger than or equal to a preset reference range, continues for a predetermined time and is continuously detected while the difference between the measured body temperature value and previously derived feature values is continuously monitored, the processor 250 may store an abnormal data section in which body temperature abnormality is generated.

In operation 727, the processor 250 may provide a notification of abnormality of body temperature of the user. The processor 250 may provide information related to abnormality of body temperature of the user to the user on the basis of a predetermined output scheme. The processor 250 may output the information related to abnormality of body temperature of the user through visual information, acoustic information, and/or tactile information. The processor 250 may transmit the information related to abnormality of body temperature of the user to the outside (for example, the electronic device of the person to measure (for example, the second electronic device 101)) through a predetermined communication scheme.

In operation 729, the processor 250 may initialize the calculated duration time. The processor 250 may initialize the calculated duration time, proceed to operation 707, and perform operations after operation 707.

According to an embodiment of the disclosure, when measuring body temperature of the user, the processor 250 may define various elements (for example, environment variables) which may influence measurement of body temperature of the user and also consider the various elements to measure body temperature.

According to an embodiment, in the operation of estimating core body temperature and when calculating basal body temperature as described with reference to FIG. 5 and/or FIG. 7, the processor 250 may additionally consider elements (for example, atmospheric temperature and/or surrounding temperature) by external factors. According to an embodiment, when body temperature of the user is measured, if atmospheric temperature (or surrounding temperature) is cool or warm, difference from core body temperature may not be large, but the atmospheric temperature may significantly influence body temperature at end parts of the body (for example, arm, leg, finger, scalp, and/or ear).

The processor 250 may acquire current atmospheric temperature information at the corresponding location from external common data (for example, weather data of the weather center) or internal common data (for example, weather data of a weather application) on the basis of location information acquired through a GNSS communication module of the first electronic device 201. According to an embodiment, when the first electronic device 201 exists in an indoor environment, the processor 250 may acquire temperature information on surrounding temperature from neighboring IoT devices (for example, an air conditioner and/or a thermometer). According to an embodiment, it is possible to estimate optimal body temperature by determining the closets environment on the basis of statistical big data information at the residence location of the user accounting for the largest portion of the day in a normal environment.

The processor 250 may control a body temperature correction value for each body part according to the type of the first electronic device 201 on the basis of the acquired information (for example, existing information, temperature information, and/or big data information) to correct the body temperature value of the user.

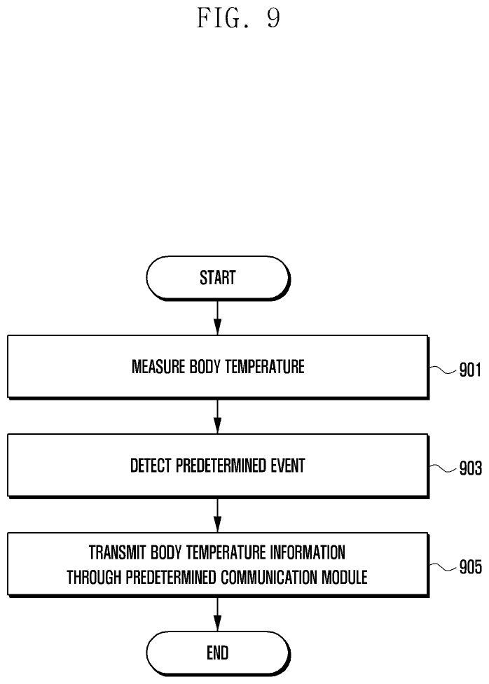

FIG. 9 is a flowchart illustrating a method of operating an electronic device according to an embodiment of the disclosure.

According to an embodiment, FIG. 9 illustrate an example of the operation for providing body temperature information measured by the first electronic device 201 (for example, the electronic device 410 or 420 of the person 415 or 425 to be measured of FIG. 4) to an external device.

Referring to FIG. 9, in operation 901, the processor 250 of the first electronic device 201 may measure body temperature of the user. The first electronic device 201 may periodically (or according to a predetermined period) measure body temperature of the user through the sensor module 210 (for example, the body thermometer 211), and the processor 250 may collect the sensor data. According to an embodiment, when power consumption is not a restraint of the first electronic device 201, the processor 250 may measure body temperature of the user at ordinary times.

In operation 903, the processor 250 may detect a predetermined event related to provision of body temperature information. According to an embodiment, when the processor 250 may event may include an abnormality of body temperature of the user (for example, the person to be measured) or detecting a request (for example, predetermined communication) from an external device (for example, the electronic device of the user to measure (for example, the second electronic device 101).

In operation 905, the processor 250 may transmit body temperature information to the external device (for example, the second electronic device 101) through a communication module. The processor 250 may transmit body temperature information to the external device through a first communication module for first communication or a second communication module for second communication different from the first predetermined communication.

According to an embodiment, when providing body temperature information measured by the first electronic device 201 to the external device, the processor 250 may perform at least one communication with the external device through the first communication or the second communication different from the first communication. The first communication may include, for example, at least one of Bluetooth, BLE, and WiFi communication. The second communication may include, for example, at least one of UWB and/or IrDA communication. When transmitting body temperature information to the external device (for example, the second electronic device 101) through the communication module, the processor 250 may provide a user interface (UI) informing of transmission of body temperature information to the external device through the output module 230.

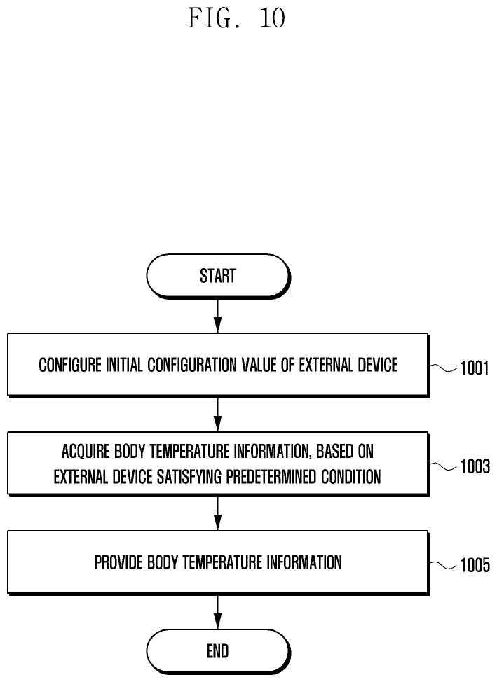

FIG. 10 is a flowchart illustrating a method of operating an electronic device according to an embodiment of the disclosure.

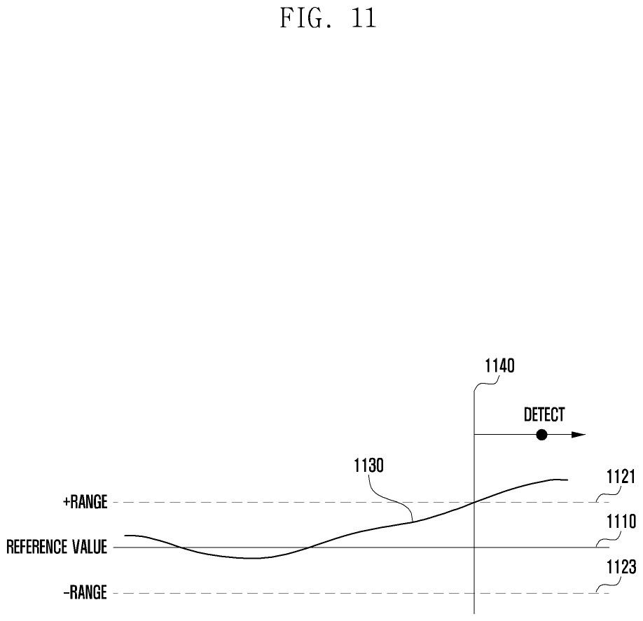

FIG. 11 illustrates an example of a reference condition configured in an external device by an electronic device according to an embodiment.

The person 435 through electronic device 101 can configured the second electronic device 101 to set a reference value and a range, and provide the electronic device 101 with the measured body temperature when the measured body temperature is outside the range from the reference value.

According to an embodiment, FIG. 10 illustrates an example of the operation in which the second electronic device 101 (for example, the electronic device 430 of the person 435 to measure of FIG. 4) collects body temperature of the person to be measured.

Referring to FIG. 10, in operation 1001, the processor 120 of the electronic device 101 may configure an initial configuration value of the external device (for example the electronic device of the person to be measured (for example, the first electronic device 201)). The initial configuration value (for example, the first predetermined threshold value of FIG. 7) may include a reference value and/or a specific range for determining whether body temperature of the person to be measured is abnormal. For example, the initial configuration value may include a reference value and/or a specific range by which the external device (for example, the first electronic device 201) may determine an abnormal body temperature change range of the person to be measured.

When initially configuring the electronic device (for example, the first electronic device 201) of the person to be measured in connection with measurement of body temperature of the person to be measured, the first electronic device 201 and the electronic device of the person to measure (for example, the second electronic device 101) may configure the initial configuration value in the first electronic device 201 through the connection of predetermined communication (for example, BLE connection). The initial configuration value may be a reference value and/or a specific range by which the first electronic device 201 may determine the abnormal body temperature change range of the person to be measured. An example thereof is illustrated in FIG. 11.

As illustrated in FIG. 11, the initial configuration value may be a value between a normal temperature reference value 1110 (for example, a reference value of about 36.5 degrees) of the person to be measured, calculated from a temperature trend of the person to be measured until the last day and a normal change range (for example, a normal range of about ±1.5 degrees) (for example, a value between a +range 1121 and a −range 1123). For example, the first electronic device 201 may monitor body temperature of the person to be measured on the basis of the initial configuration value and determine abnormality of body temperature of the user at a time point 1140 at which the measured body temperature value 1130 escapes the normal change range.

In operation 1003, the processor 120 may acquire the corresponding body temperature information of the person to be measured on the basis of the external device (for example, the first electronic device 201) satisfying a predetermined condition. The processor 120 may receive body temperature information from the external device intended (or designated) by the user (for example, the person to measure) or the external device having abnormality of body temperature through a predetermined communication module. The operation in which the second electronic device 101 acquires body temperature information from the first electronic device 201 according to a predetermined condition is described with reference to the following drawings.

In operation 1005, the processor 120 may provide body temperature information to the user (for example, the person to measure). The processor 120 may provide the body temperature information acquired from the external device to the user in a predetermined scheme. The processor 120 may output the body temperature information acquired from the external device through visual information, acoustic information, and/or tactile information.

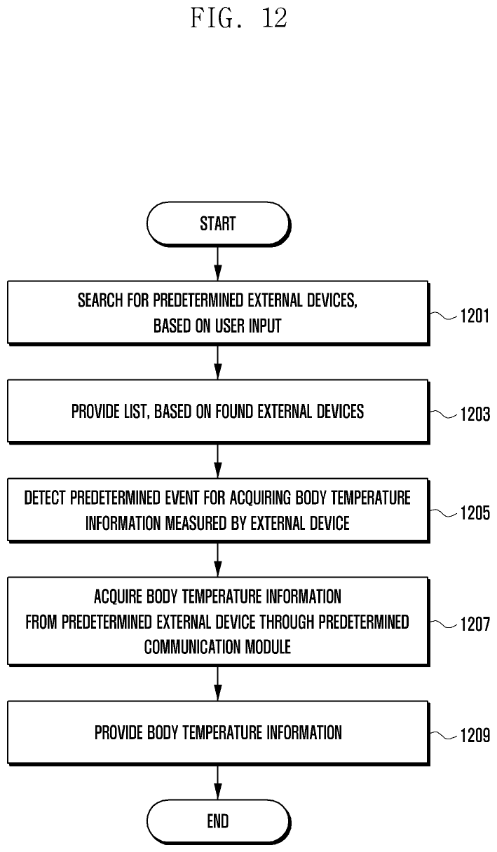

FIG. 12 is a flowchart illustrating a method of operating an electronic device according to an embodiment of the disclosure.

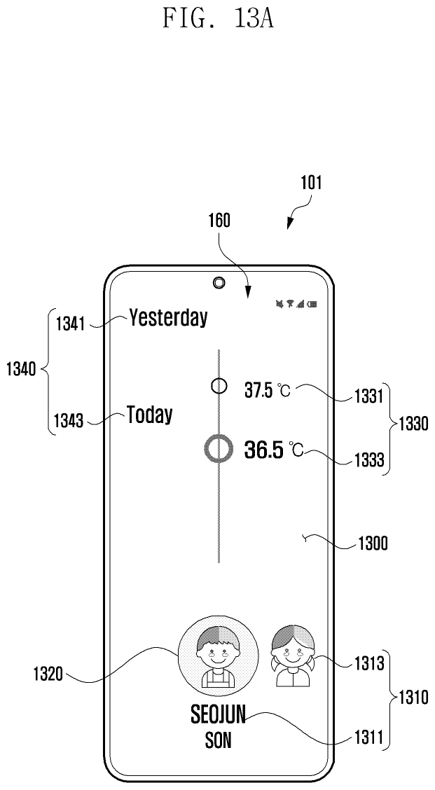

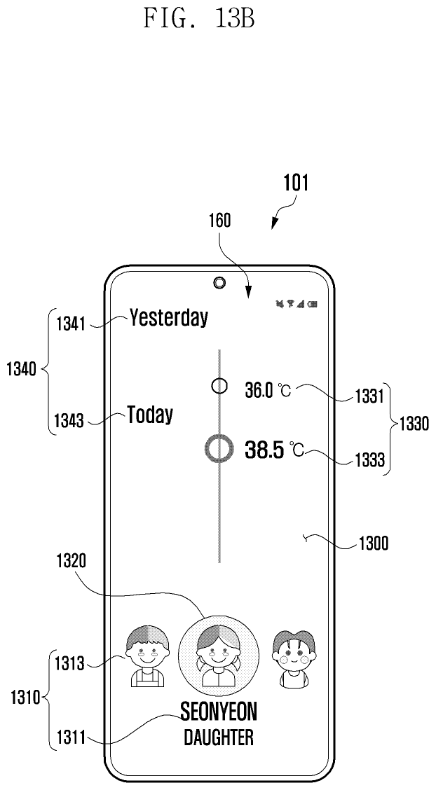

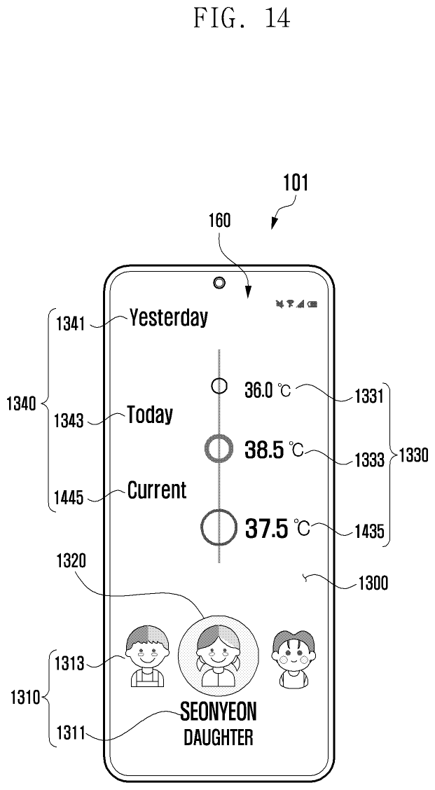

FIGS. 13A and 13B and FIG. 14 illustrate various examples of a user interface for providing body temperature information in an electronic device according to an embodiment of the disclosure.

According to an embodiment, FIG. 12 illustrates an example of the operation in which the second electronic device 101 (for example, the electronic device 430 of the person 435 to measure of FIG. 4) designates a person to be measured, collect the body temperature, and provides the same to a user (for example, a person to measure).

Referring to FIG. 12, in operation 1201, the processor 120 of the electronic device 101 may search for at least one neighboring external device (for example, electronic devices (for example, the first electronic device 201) of the person to be measured) on the basis of a user input. The user input may include an input of executing a predetermined application for managing body temperature. The user input may include various inputs (for example, a voice input, a motion input of a device, a user gesture input, a function button (for example, a physical button or a software button) input, and/or an input using an electronic pen) designated to execute a predetermined application. In response to a particular user input, the processor 120 may search for neighboring external devices (for example, at least one first electronic device 201) using a first communication, for example, at least one of, Bluetooth, BLE, and WiFi communication).

In operation 1203, the processor 120 may generate and provide a list on the basis of at least one found external device. The list may be provided in the form of a list including predetermined objects (for example, text objects and/or icon objects indicating device information and/or user information) related to each of the at least one found external devices and displayed on the display. In certain embodiments, the list may be provided through a user interface including the objects related to each of the at least one found external devices. In certain embodiments, the user can select one of the icon objects to select a person 415, 425 for measuring the body temperature. According to an embodiment, an example of the user interface providing body temperature information is illustrated in FIGS. 13A and 13B.

Referring to FIGS. 13A and 13B, a user interface 1300 for providing body temperature information may be provided through the display module 160 of the electronic device 101. The user interface 1300 may include a user information area 1310 providing information related to at least one person to be measured, a designation area 1320 for designating a target whose body temperature is to be measured among at least one person to be measured, and result areas 1330 and 1340 for providing body temperature information related to the person to be measured, designated through the designation area 1320 and a history thereof. In an embodiment, the result areas 1330 and 1340 may include, for example, a body temperature information area 1330 for displaying body temperature information and a time information area 1340 for providing time information corresponding to body temperature information.

The user information area 1310 may include a profile (for example, a name and/or a nickname) object 1311 related to the user (for example, the person to be measured) and an image (for example, a profile picture) object 1313. The user information area 1310 may receive a first user input (for example, flick or swipe) and provide screen scroll in a predetermined direction (for example, a left and right direction) within the user information area 1310 according to the first user input. The target whose body temperature is to be measured may be designated by a touch input on the object 1313.

The body temperature information area 1330 and the time information area 1340 may include body temperature information 1331 and 1333 previously measured for targets whose body temperature is to be measured, located in the designation area 1320 and time information 1341 and 1343 related to measurement of the body temperature information 1331 and 1333. The body temperature information 1331 and 1333 may be mapped to the time information 1341 and 1343.

FIG. 13A illustrates an example in which a first person to be measured is located in the designation area 1320 and body temperature information related to the first person to be measured is provided. FIG. 13B illustrates an example in which a second person to be measured is located in the designation area 1320 and body temperature information related to the second person to be measured is provided.

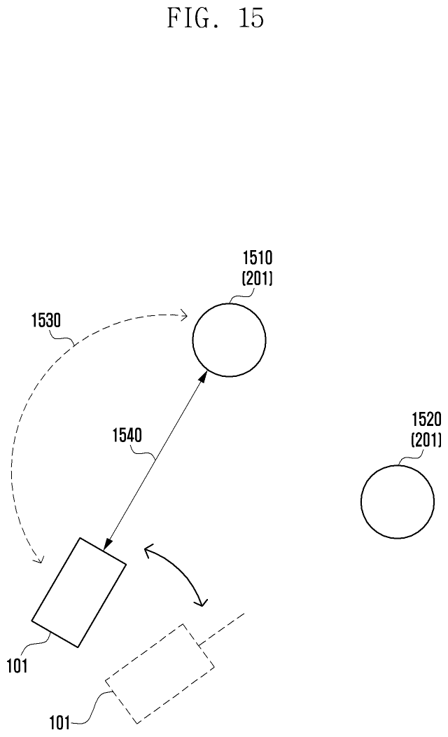

Referring back to FIG. 12, in operation 1205, the processor 120 may detect a predetermined event (or trigger) for acquiring body temperature information measured by the external device. When an object is selected from the list, the processor 120 may transmit a body temperature information request to the external device designated using the first communication. Using the first communication or the second communication for a body temperature information request, the processor 120 may transmit the body temperature information request to the external device designated (for example, in a predetermined distance and direction) through a predetermined communication (for example, the second predetermined communication) via a predetermined communication module (for example, the second predetermined communication module). An example of the operation for designating the external device through the predetermined communication (for example, the second predetermined communication) is described with reference to FIG. 15.

In operation 1207, the processor 120 may acquire body temperature information from the designated external device through a predetermined communication module. The processor 120 may receive body temperature information from the external device designated by the user (for example, the person to measure) through the communication module. The processor 120 may receive body temperature information of the person to be measured from the external device through the first communication and/or the second communication. The operation in which the second electronic device 101 acquire body temperature information from the first electronic device 201 through the communication module is described with reference to the following drawings.

In operation 1209, the processor 120 may provide body temperature information. The processor 120 may display body temperature information acquired from the external device on a user interface to provide the same to the user. According to an embodiment, an example of the user interface providing body temperature information is illustrated in FIG. 14.

Referring to FIG. 14, the user interface 1300 for providing body temperature information according to an embodiment may correspond to, for example, the user interface 1300 described with reference to FIGS. 13A and 13B. According to an embodiment, compared to FIGS. 13A and 13B, in additional to the user interface 1300, body temperature information 1435 acquired from the external device of the target whose body temperature is to be measured (for example, the person to be measured displayed through the designation area 1320) and time information 1445 for measurement of the body temperature information 1435 may be further provided in FIG. 14. The body temperature information 1435 and the time information 1445 may be accumulated and provided. According to an embodiment, result areas (for example, the body temperature information area 1330 and the time information area 1340) may receive a second user input (For example, a flick or swipe input) and provide screen scroll in a predetermined direction (for example, an up and down direction) within the result areas according to the second user input. The body temperature information and the time information in the result areas may be scrolled on the basis of the second user input.

FIG. 15 illustrates an example of the operation for performing communication between electronic devices according to an embodiment of the disclosure.

According to an embodiment, FIG. 15 illustrates an operation in an environment in which electronic devices 1510 and 1520 (for example, the electronic device 201) of a plurality of people to be measured and the electronic device 101 (for example, the second electronic device 101) of a person to measure coexist within a predetermined space (for example, home). According to an embodiment, FIG. 15 illustrates an example of an operation in which the person to measure designates the first electronic device 210 (for example, an electronic device 1510 of FIG. 15) of the person to be measured through predetermined communication by using the second electronic device 101 to designate one person to be measured who is a target whose body temperature is to be measured among the plurality of people to be measured.

The second electronic device 101 and the electronic devices 1510 and 1520 (for example, the first electronic device 201) of the plurality of people to be measured may recognize each other in a predetermined space (or limited space) within a predetermined range (or distance) (for example, within a radius of 10 m) through first communication 1530, for example, at least one of Bluetooth, BLE, and WiFi communication). For example, the first communication 1530 may be a communication standard that is short in range, such as short range communications. For example, the first predetermined communication 1530 may include at least one of Bluetooth, BLE, and/or WiFi communication for establishing a first network for the designated range. The first communication 1530 may be used for initially identifying the coexistence of the electronic devices 1510 and 1520 (for example, the first electronic device 201) of the people to be measured and the second electronic device 101 of the person to measure in the same space.

In order to acquire body temperature of designated targets among a plurality of person to be measured, the person to measure may use second communication 1540, for example, at least one of UWB and/or IrDA communication, for determining a relative location (or direction and distance) between the person to measure and the people to be measured. For example, the second predetermined communication may indicate communication for designating a target whose body temperature is to be measured. For example, the second communication may include UWB and/or IrDA communication. The electronic devices 1510 and 1520 (for example, the first electronic device 201) of the people to be measured and the second electronic device 101 of the person to measure may recognize the coexistence there of in a predetermined space through the first predetermined communication, and may determine (or select) one electronic device (for example, the electronic device 1510) among the electronic devices 1510 and 1520 of the people to be measured through the second predetermined communication to designate the person to be measured for body temperature measurement.

Referring to the example of FIG. 15, the second electronic device 101 of the person to measure and the electronic devices 1510 and 1520 of the people to be measured may be connected through the first communication (for example, BLE) in a predetermined space. The person to measure may make the second electronic device 101 face a direction in which the target whose body temperature is to be measured is located (for example, a direction of the electronic device 1510). The second electronic device 101 may acquire body temperature information of the person to be measured from the electronic device 1510 of the corresponding person to be measured through communication with the electronic device (for example, the electronic device 1510) of the person to be measured located within a predetermined distance and/or located in a predetermined direction among the electronic devices 1510 and 1520 of the plurality of people to be measured existing in the predetermined space through the second predetermined communication.

The electronic device 1510 of the corresponding person to be measured may detect initiation of the operation of measuring body temperature of the person to be measured through the second communication with the second electronic device 101 and transmit body temperature information of the person to be measured to the second electronic device 101 through the first communication and/or the second communication.

According to an embodiment, as illustrated in FIG. 15, the second electronic device 101 of the person to measure and the electronic devices 1510 and 1520 (for example, the first electronic device 201) of the people to be measure may perform and provide body temperature measurement on the basis of the predetermined (or limited) distance and/or direction.

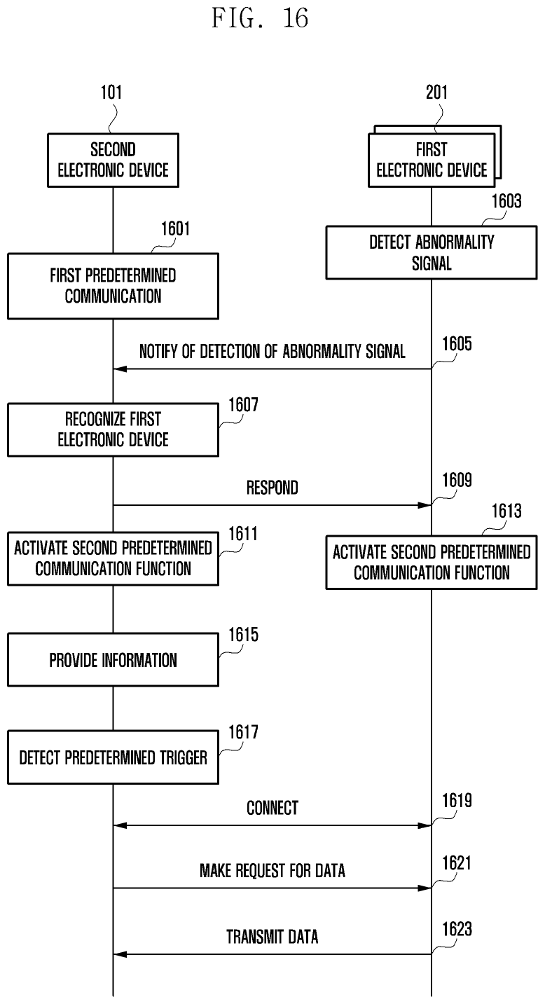

FIG. 16 illustrates an example of an operation for providing body temperature information through communication between electronic devices according to an embodiment of the disclosure.

According to an embodiment, FIG. 16 illustrates an example of an operation in which the first electronic device 201 (for example, the electronic device of the person to be measured) measures body temperature of the user (for example, the person to be measured) and provide the measured body temperature to the second electronic device 101 (for example, the electronic device of the person to measure) by a predetermined condition (for example, detection of abnormality of body temperature). For example, FIG. 16 shows an example of an operation for providing body temperature information of the person to be measured through communication with the second electronic device 101 on the basis of detection of a predetermined event such as detection of abnormality from body temperature measured by the first electronic device 201.

Referring to FIG. 16, in operation 1601, the second electronic device 101 (for example, the electronic device of the person to measure) may perform first communication with the first electronic device 201 (for example, the electronic device at least one person to be measured). The second electronic device 101 may be performing BLE scan in order to receive an advertising packet (for example, a BLE advertising message) which may be generated at a predetermined time point from at least one neighboring first electronic device 201. For example, the first electronic device 201 may periodically broadcast a BLE advertising message. In an embodiment, the BLE advertising message may include device information of the first electronic device 201. The second electronic device 101 may perform periodic BLE scan and, when receiving the BLE advertising message of the first electronic device 201, make the connection with the first electronic device 201.

In operation 1603, the first electronic device 201 may detect an abnormality signal. The first electronic device 201 may continuously monitor difference between a body temperature value measured for the user (for example, the person to be measured) and previously derived feature values and detect an abnormality signal continuously detected due to the state in which the difference between the body temperature value and the feature value is larger than predetermined reference difference for a predetermined time during the monitoring.

In operation 1605, the first electronic device 201 may provide a notification of detection of the abnormality signal to the second electronic device 101 through the first communication on the basis of detection of the abnormality signal. The first electronic device 201 may broadcast an advertising packet including information related to detection of the abnormality signal. For example, the first electronic device 201 may provide a notification of abnormality of body temperature of the user (for example, the person to be measured) to the person to measure through the first communication (for example, the advertising packet).

In operation 1607, the second electronic device 101 may receive the advertising packet including information related to detection of the abnormality signal from the first electronic device 201 and recognize the first electronic device 201 on the basis of reception of the corresponding advertising packet. For example, since the second electronic device 101 is continuously performing BLE scan, the second electronic device may recognize which electronic device detects the abnormality signal among the neighboring first electronic devices 201 on the basis of device information of the advertising packet.

In operation 1609, the second electronic device 101 may transmit a response corresponding to reception of the advertising packet including information related to detection of the abnormality signal from the first electronic device 201 to the first electronic device 201 through communication. The second electronic device 101 may transmit a response corresponding to reception of the advertising packet to the first electronic device 201 through the first communication. The first electronic device 201 and the second electronic device 101 may transmit and receive a notification and a response according to detection of the abnormality signal through other communication (for example, legacy communication for example, 3G communication and/or 4G communication), 5G communication, or next-generation communication).

In operation 1611, the second electronic device 101 may activate a relevant function for the second communication with the first electronic device 201 on the basis of reception of the advertising packet including information related to detection of the abnormality signal from the first electronic device 201. According to an embodiment, when recognizing the first electronic device 201 and the abnormality signal generated thereby, the second electronic device 101 may activate (or turn on) a communication module (for example, the second communication module 312 of FIG. 3) for performing UWB communication with the corresponding first electronic device 201.

In operation 1613, the first electronic device 201 may activate a relevant function for the second communication with the second electronic device 101. The first electronic device 201 may activate (or turn on) the communication module (for example, the second communication module 222 of FIG. 2) for performing UWB communication with the second electronic device 101 on the basis of reception of the response from the first electronic device 201. According to an embodiment, operation 1611 and operation 1613 are not limited to the illustrated sequences and may be performing in parallel (or substantially at the same time), inversely sequentially, or heuristically.

In operation 1615, the second electronic device 101 may provide information related to abnormality of body temperature of the person to be measured to the user (for example, the person to measure) of the second electronic device 101. The second electronic device 101 may inform the user of detection of the first electronic device 201 detecting abnormality of body temperature through a predetermined output scheme. The second electronic device 101 may output information related to abnormality of body temperature of the person to be measured through visual information, acoustic information, and/or tactile information on the basis of a predetermined scheme. For example, the second electronic device 101 may display device information of the first electronic device 201 transmitting information related to detection of the abnormality signal and/or user information corresponding to the first electronic device 201 on a display module (for example, the display module 160 of FIG. 1). Accordingly, the second electronic device 101 may inform the person to measure which person to be measured has the first electronic device 201 detecting the abnormality signal.

According to an embodiment, for example, when the number of people to be measured is plural, the second electronic device 101 may display information related to the location and/or direction of the first electronic device 201 detecting abnormality of body temperature (for example, the current location and/or direction of the person to be measured) in a visually distinguished manner. For example, the second electronic device 101 may distinguish (or highlight) (for example, change a color and/or size) an object related to the person to be measured of the first electronic device 201 detecting abnormality of body temperature, display the object to be different from another person to be measured, and provide the same.

The user may identify, through the second electronic device 101, which person to be measured has the first electronic device 201 from which the notification is currently received. According to an embodiment, when the user identifies a corresponding notification through the second electronic device 101, the user may visually identify situation information (or state) related to the corresponding neighboring person to be measured. For example, the user may identify various pieces of situation information indicating that the corresponding person to be measured is eating something, the person to be measured is exercising, and/or surrounding temperature. For example, the user may identify in advance whether there is at least one element which may influence body temperature of the person to be measured.

In operation 1617, the second electronic device 101 may detect a predetermined trigger (or operation event). The second electronic device 101 may measure a predetermined distance and direction (for example, performing ranging) for the first electronic device 201 through the second communication using the communication module (for example, the second communication module 312 of FIG. 3). The second electronic device 101 may continuously perform ranging while an application for providing a body temperature measurement function is executed.

The user may make the second electronic device 101 face the corresponding person to be measured (for example, the first electronic device 201) by controlling the second electronic device 101. According to an embodiment, when it is determined that there is no at least one element which may influence body temperature of the corresponding person to be measured and that there is a need to measure body temperature of the corresponding person to be measured on the basis of the result of identifying surrounding situation information, the user may perform an operation (for example, a user input) making the second electronic device 101 face (or head for) the person to be measured (for example, the first electronic device 201). According to an embodiment, when information related to abnormality of body temperature is provided and then a user input is detected within a predetermined time, the second electronic device 101 may process the user input as triggering for the second communication.

According to an embodiment, when it is determined that there is no at least one element which may influence body temperature of the corresponding person to be measured and that there is no need to measure body temperature of the corresponding person to be measured on the basis of the result of identifying situation information, the user may not perform an additional operation (for example, controlling the second electronic device 101). According to an embodiment, when a user input is not detected from the user for a predetermined time after detection of the abnormality signal from the first electronic device 201 and/or activation of the second communication function, the second electronic device 101 may deactivate the second communication function and initialize the operation related to collection of body temperature information of the person to be measured.

In operation 1619, the second electronic device 101 may be in the state in which the second communication function is activated and may be connected to the first electronic device 201 through the second communication. The second electronic device 101 may be connected to the first electronic device 201 in a predetermined direction and distance on the basis of the second communication. The second electronic device 101 may perform a predetermined authentication operation when the distance and/or direction between the second electronic device 101 and the first electronic device 201 satisfies a predetermined reference (or condition). For example, the second electronic device 101 may perform an authentication operation for acquiring and providing personal privacy information (for example, body temperature information) of the first electronic device 201.

In operation 1621, the second electronic device 101 may make a request for data (for example, body temperature information) to the first electronic device 201 on the basis of the second communication. According to an embodiment, when authentication with the first electronic device 201 is completed, the second electronic device 101 may make a request for body temperature information to the first electronic device 201. The second electronic device 101 may make a request for data (for example, body temperature information) through other communication (for example, BLE communication, legacy communication (for example, 3G communication and/or 4G communication), 5G communication, and/or next-generation communication) with the first electronic device 201 in a predetermined distance and direction by the second communication.

When receiving the request for data from the second electronic device 101 through the second communication, the first electronic device 201 may provide the data (for example, body temperature information) to the second electronic device 101 through communication (for example, the first communication, the second communication, or other communication) in operation 1623. The first electronic device 201 may collect body temperature information acquired through the body temperature measurement function or pre-measured body temperature information and transmit the collected body temperature information to the second electronic device 101.

The first electronic device 201 may transmit data (for example, body temperature information) through other communication (for example, legacy communication (for example, 3G communication and/or 4G communication), 5G communication, and/or next-generation communication) to the second electronic device 101 in a predetermined distance and direction by the second communication. The first electronic device 201 may transmit data (for example, body temperature information) through the second communication. For example, the first electronic device 201 may transmit data in an independent session separated from ranging of the second communication to the second electronic device 101 in a predetermined distance and direction through ranging using the second communication.

The second electronic device 101 may receive data from the first electronic device 201 through the second communication, display the received data (for example, body temperature information) on the display module 160, and provide the same to the user. The user (for example, the person to measure) may approach close to the corresponding person to be measured to detect a detailed state on the basis of body temperature information acquired by the second electronic device 101 from the first electronic device 201 or take additional action of re-measuring body temperature of the person to be measured through a more precise body temperature measurement device.

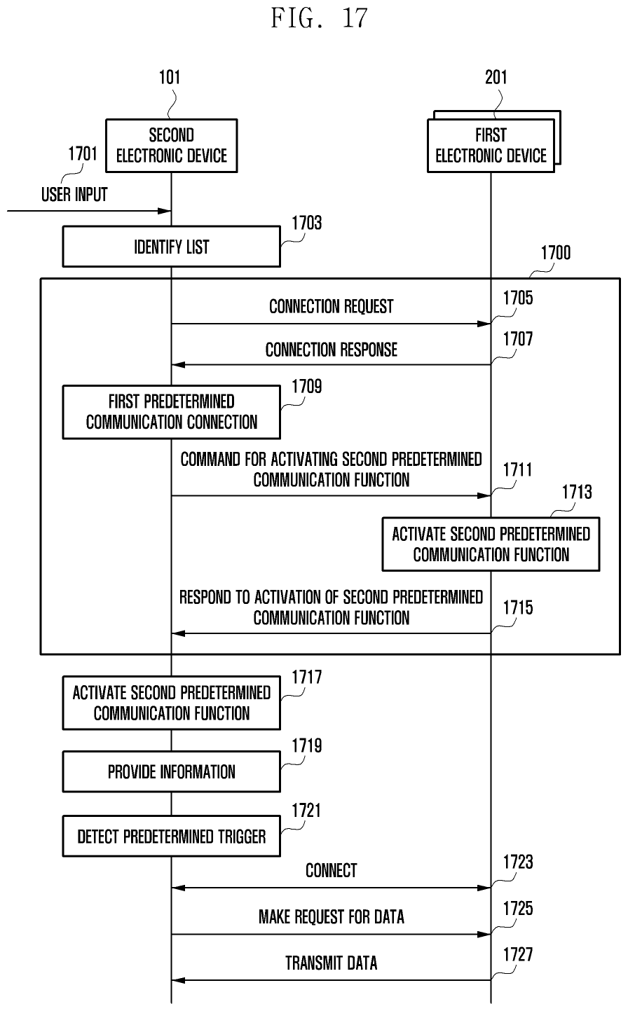

FIG. 17 illustrates an example of an operation for providing body temperature information through communication between electronic devices according to an embodiment of the disclosure.

According to an embodiment, FIG. 17 illustrates an example of an operation in which the second electronic device 101 (for example, the electronic device of the person to measure) provides body temperature information of the person to be measured through communication with the first electronic device 201 on the basis of a request from the user (for example, the person to measure). For example, FIG. 17 shows an example of an operation of allowing the first electronic device 201 of the person to be measured around the user (for example, the second electronic device 101) to prepare communication and then providing body temperature information of the predetermined person to be measured from the predetermined first electronic device 201 through the communication on the basis of a user input for acquiring the body temperature information of the person to be measured. For example, FIG. 17 shows an example of, when the user (for example, the person to measure) desires to acquire body temperature information from the first electronic device 201 of the person to be measured at a specific time point, acquiring and providing body temperature information through the communication by using the second electronic device 101.

Referring to FIG. 17, in operation 1701, the user (for example, the person to measure) may make a user input related to an intention to measure body temperature of the person to be measured by using the second electronic device 101. According to an embodiment, when a situation in which the user desires to identify body temperature of the person to be measured occurs periodically and/or at any time point, a signal corresponding to the measurement intention may be input into the second electronic device 101. The user input may include various inputs designated to execute a predetermined application. For example, the user input may include a voice command input, a motion input of a device, a user gesture input, a function button (for example, a physical button or a software button) input, and/or an input using an electronic pen.

In operation 1703, the second electronic device 101 may identify a list. The second electronic device 101 may identify a list related to at least one first electronic device 201 (or paired first electronic device 201) around the second electronic device 101 on the basis of a user input. According to an embodiment, when detecting a user input, the second electronic device 101 may search (or scan) for neighboring external devices (for example, at least one first electronic device 201) on the basis of first communication (for example, Bluetooth, BLE, and/or WiFi communication) and identify a list of found first electronic devices 201.

According to an embodiment, when detecting a user input, the second electronic device 101 may recognize a user intention according to the user input and display relevant information (for example, a user interface related to body temperature measurement) through the display module 160. According to some embodiments, the second electronic device 101 may provide a sound and/or a vibration output in response to recognition of the user intention.

In operation 1700, the second electronic device 101 and the first electronic device 201 (for example, the first electronic device 201 found (or registered in the list) may perform an operation for activating the second communication function sequentially or in parallel. The operation (for example, operation 1700) for activating the second communication function may be performed including operations 1705 to 1715. For example, operation 1700 may be separately performed for each of the corresponding first electronic devices 201 in accordance with the number of first electronic devices 201 (for example, the first electronic device 201 found (or registered in the list) of people to be measured around the second electronic device 101.

In operation 1705, the second electronic device 101 may transmit a first communication connection request (for example, a BLE connection request) for the first communication connection with the first electronic device 201 to the first electronic device 201. For example, the second electronic device 101 may perform an operation for the BLE communication connection with the first electronic device 201.

In operation 1707, the first electronic device 201 may receive the first communication connection request from the second electronic device 101 and transmit a first communication connection response (for example, a BLE connection response) corresponding to the first communication connection to the second electronic device 101. For example, the first electronic device 201 may perform an operation for the BLE communication connection with the second electronic device 101.

In operation 1709, the second electronic device 101 may make the first communication connection (for example, the BLE connection) with the first electronic device 201 on the basis of reception of the first communication connection response from the first electronic device 201.

In operation 1711, the second electronic device 101 may transmit a command (for example, a UWB on command) making a request for activating the second communication function to the first electronic device 201 through the first communication on the basis of the first communication connection with the first electronic device 201. For example, the second electronic device 101 may transmit a command for activating (or turning on) a communication module (for example, the second communication module 222 of FIG. 2) to perform the second communication (for example, UWB) with the first electronic device 201. The first electronic device 201 and the second electronic device 101 may transmit and receive the command for activating the second communication function through other communication (for example, legacy communication (for example, 3G communication and/or 4G communication), 5G communication, or next-generation communication).

In operation 1713, the first electronic device 201 may receive the command for activating the second communication function from the second electronic device 101 through the first communication and activate the second communication function on the basis of reception of the command. The first electronic device 201 may activate (or turn on) the communication module (for example, the second communication module 222 of FIG. 2) to perform UWB communication with the second electronic device 101.

In operation 1715, the first electronic device 201 may transmit a response according to activation of the second communication function to the first electronic device 201 through the first communication. The first electronic device 201 may activate the second communication function and transmit a response including information indicating completion of activation of the second communication function to the second electronic device 101.

According to an embodiment, when identifying completion of activation of the second communication function from the first electronic device 201 through the first communication, the second electronic device 101 may perform operation 1700 for the next first electronic device 201 registered in the list. For example, the second electronic device 101 may perform the operation for activating the second communication function for a plurality of first electronic devices 201 registered in the list sequentially or in parallel. The operation (for example, operation 1700) for activating the second communication function may be performed separately for each of the corresponding first electronic devices 201 in accordance with the number of first electronic devices 201 (for example, found or registered in the list) of people to be measured around the second electronic device 101.

According to an embodiment, when completing the performance in connection with operation 1700 for all the first electronic devices 201 registered in the list, the second electronic device 101 may identify whether operation is successful.

According to an embodiment, in connection with operation 1700, the second electronic device 101 may display a list related to the first electronic devices 201 of neighboring people to be measured, perform operation 1700 with one predetermined first electronic device 201 selected (or designated) by the user, and proceed to operation 1717 directly after operation 1700.

The second electronic device 101 may operate as an advertiser for the first communication and broadcast an advertising packet (for example, a BLE advertising packet) including a command (for example, UWB on command) for activating the second communication function. According to an embodiment, at least one first electronic device 201 around the second electronic device 101 may perform periodic BLE scan, receive the advertising packet from the second electronic device 101, activate the second communication function, and transmit a response corresponding thereto to the second electronic device 101.

When receiving the response according to activation of the second communication function from the first electronic device 201 through the first communication, the second electronic device 101 may activate the second communication function in operation 1717. The second electronic device 101 may activate (or turn on) the communication module (for example, the second communication module 312 of FIG. 3) to perform UWB communication with the first electronic device 201.

In operation 1719, the second electronic device 101 may provide information indicating that the second communication with the first electronic device 201 is possible to the user (for example, the person to measure) of the second electronic device 101. The second electronic device 101 may output information (for example, information related to completion of preparation of body temperature measurement) indicating that body temperature of the person to be measured can be measured through visual information, acoustic information, and/or tactile information on the basis of a predetermined output scheme. For example, the second electronic device 101 may display device information of the first electronic device 201 capable of performing the second communication and/or user information corresponding to the first electronic device 201 through the display module 160.

In operation 1721, the second electronic device 101 may detect a predetermined trigger (or operation event). The second electronic device 101 may perform measurement (for example, ranging) a predetermined distance and direction for the first electronic device 201 through the second communication using the communication module (for example, the second communication module 312 of FIG. 3). The second electronic device 101 may continuously perform ranging while an application for providing a body temperature measurement function is executed.

The user may determine a target user (for example, the person to be measured) (or the first electronic device 201 corresponding to the user) whose body temperature is to be measured on the basis of information provided through the second electronic device 101. The user may identify people to be measured (or the first electronic devices 201 of the people to be measured) whose body temperature can be measured on the basis of information displayed through the display module 160 of the second electronic device 101 and determine at least one targets whose body temperature is to be measured among the identified people to be measured.

According to an embodiment, when the user identifies a corresponding notification through the second electronic device 101, the user may visually identify situation information (or state) related to the corresponding neighboring person to be measured. For example, the user may identify various pieces of situation information indicating that the corresponding person to be measured is eating something, the person to be measured is exercising, and/or surrounding temperature. For example, the user may identify in advance whether there is at least one element which may influence body temperature of the person to be measured.

The user may make the second electronic device 101 face the corresponding person to be measured (for example, the first electronic device 201) by controlling the second electronic device 101. The user may perform an operation (for example, a user input) for making the second electronic device 101 face (or head for) the person to be measured (for example, the first electronic device 201) determined that body temperature is needed to be measured. According to an embodiment, when the second electronic device 101 activates the second communication function and then detects a user input, the second electronic device 101 may process the user input as triggering for the second communication.

In operation 1723, the second electronic device 101 may be in the state I which the second communication function is activated and may be connected to the first electronic device 201 through the second communication. The second electronic device 101 may be connected to the first electronic device 201 in a predetermined direction and distance on the basis of the second communication. The second electronic device 101 may perform a predetermined authentication operation when the distance and/or direction between the second electronic device 101 and the first electronic device 201 satisfies a predetermined reference (or condition). For example, the second electronic device 101 may perform an authentication operation for acquiring and providing personal privacy information (for example, body temperature information) of the first electronic device 201.

In operation 1725, the second electronic device 101 may make a request for data (for example, body temperature information) to the first electronic device 201 on the basis of the second communication. According to an embodiment, when authentication with the first electronic device 201 is completed, the second electronic device 101 may make a request for body temperature information to the first electronic device 201. The second electronic device 101 may make a request for data (for example, body temperature information) through other communication (for example, BLE communication, legacy communication (for example, 3G communication and/or 4G communication), 5G communication, and/or next-generation communication) with the first electronic device 201 in a predetermined distance and direction by the second communication.

When receiving the request for data from the second electronic device 101 through the second communication, the first electronic device 201 may provide the data (for example, body temperature information) to the second electronic device 101 through communication (for example, the first communication, the second communication, or other communication) in operation 1727. The first electronic device 201 may collect body temperature information acquired through the body temperature measurement function or pre-measured body temperature information and transmit the same to the second electronic device 101.

The first electronic device 201 may transmit data (for example, body temperature information) through other communication (for example, legacy communication (for example, 3G communication and/or 4G communication), 5G communication, and/or next-generation communication) with the second electronic device 101 in a predetermined distance and direction by the second communication. According to some embodiments, the first electronic device 201 may transmit data (for example, body temperature information) through the second communication. For example, the first electronic device 201 may transmit data in an independent session separated from ranging of the second communication to the second electronic device 101 in a predetermined distance and direction through ranging using the second communication.

The second electronic device 101 may receive data from the first electronic device 201 through the second communication, display the received data (for example, body temperature information) on the display module 160, and provide the same to the user. The user (for example, the person to measure) may approach close to the corresponding person to be measured to detect a detailed state on the basis of body temperature information acquired by the second electronic device 101 from the first electronic device 201 or take additional action of re-measuring body temperature of the person to be measured through a more precise body temperature measurement device.

The user may make the second communication possible between the second electronic device 101 and the first electronic device 201 through the operation for making the second electronic device 101 face the person to be measured whose body temperature is to be measured within a predetermined distance and may immediately acquire and identify body temperature information of the person to be measured through the second communication.

According to certain embodiments, an electronic device comprises: a sensor module; a communication module; and a processor operatively connected to the sensor module and the communication module, wherein the processor is configured to: measure a body temperature of a user through the sensor module, thereby resulting in a measured body temperature, estimate a core body temperature of the user, based on the measured body temperature, and provide body temperature information of the user, based on the core body temperature, wherein the measured body temperature comprises a shell temperature of a body part making contact with the electronic device, and wherein the core body temperature comprises a core temperature of an internal organ of a body of the user.

According to certain embodiments, the processor is configured to estimate the core body temperature by correcting the measured body temperature, based on a predefined body temperature correction value for a body part on which the electronic device is worn.

According to certain embodiments, the processor is configured to detect a request from an external device to provide the body temperature information, and provide the body temperature information to the external device through the communication module.

According to certain embodiments, the processor is configured to provide body temperature information to the external device through a first communication module for first communication or a second communication module for second communication different from the first communication, the first communication comprises at least one of Bluetooth, BLE, and WiFi communication, and the second communication comprises at least one of UWB and IrDA communication.

According to certain embodiments, the electronic device comprises a wearable device, the wearable device selected from a group consisting of a watch, ring, and ear phone.

An operation method performed by an electronic device (for example, the first electronic device 201 of FIG. 2) according to certain embodiments of the disclosure may include an operation of measuring body temperature of a user through a sensor module of the electronic device, thereby resulting in a measured body temperature, estimating core body temperature of the user, based on the measured body temperature, providing body temperature information of the user, based on the core body temperature, wherein the measured body temperature includes shell temperature of a body part making contact with the electronic device; and wherein the core body temperature includes the core temperature of an internal organ of the body of the user.

According to an embodiment, the operation of estimating the core body temperature may include an operation of estimating the core body temperature by correcting the measured body temperature on the basis of a predefined body temperature correction value for a body part on which the electronic device is worn.

According to an embodiment, the operation of providing the body temperature information may include an operation of detecting a request from an external device to provide body temperature information and an operation of providing the body temperature information to an external device through a first communication module of the electronic device.

According to an embodiment, the operation of providing the body temperature information may include an operation of providing body temperature information to the external device through a first communication module for first communication or a second communication module for second communication different from the first predetermined communication, the first communication may include at least one of Bluetooth, BLE, and WiFi communication, and the second predetermined communication may include at least one of UWB and IrDA communication.

According to an embodiment, the operation of detecting the predetermined event may include an operation of determining the predetermined event related to provision of body temperature information on the basis of detection of abnormality of body temperature of the user or detection of a request from the external device.

According to an embodiment, the electronic device may include wearable devices such as watch type, ring type, or in-ear type devices.

An operation method performed by an electronic device (for example, the second electronic device 101 of FIG. 3) according to certain embodiments of the disclosure may include an operation of searching for at least one neighboring external device, in response to a user input, an operation of providing a list, based on at least one found external device on a display module, an operation of detecting an event for acquiring body temperature information, an operation of acquiring body temperature information from a selected one of the at least one neighboring external device, and an operation of providing the body temperature information through a display module.

According to an embodiment, the operation of acquiring the body temperature information may include an operation of transmitting a body temperature information request to the external device through a first communication module for first communication or a second communication module for second communication different from the first communication, the first communication may include Bluetooth, BLE, and/or WiFi communication, and the second communication may include UWB and/or IrDA communication.

According to an embodiment, the operation of acquiring the body temperature information may include an operation of recognizing at least one external device existing in a predetermined space within a predetermined range through first communication module, designating the range, an operation of designating an external device of a target whose body temperature is to be measured in a predetermined direction and/or distance among the at least one external device through by a second communication module, designating the target whose body temperature is to be measured, and an operation of acquiring the body temperature information from the designated external device through the second communication module.

Certain embodiments of the disclosure illustrated in the specification and drawings only present specific examples to easily describe the technical content of the disclosure and help understanding of the disclosure but do not limit the scope of the disclosure. Accordingly, the scope of the disclosure should be construed that all modifications or modified forms derived based on the technical idea of the disclosure are included in the scope of the disclosure.

1. An electronic device comprising:
a sensor module;
a communication module; and
a processor operatively connected to the sensor module and the communication module,
wherein the processor is configured to:
measure a body temperature of a user through the sensor module, thereby resulting in a measured body temperature,
estimate a core body temperature of the user, based on the measured body temperature, and
provide body temperature information of the user, based on the core body temperature,
wherein the measured body temperature comprises a shell temperature of a body part making contact with the electronic device, and
wherein the core body temperature comprises a core temperature of an internal organ of a body of the user.

2. The electronic device of claim 1, wherein the processor is configured to estimate the core body temperature by correcting the measured body temperature, based on a predefined body temperature correction value for a body part on which the electronic device is worn.

3. The electronic device of claim 1, wherein the processor is configured to:
detect a predetermined event related to provision of the body temperature information, and
provide the body temperature information to an external device through the communication module,
wherein the predetermined event comprises the body temperature of the user or detection of a request from the external device.

4. The electronic device of claim 3, wherein the processor is configured to provide body temperature information to the external device through a first communication module for first communication or a second communication module for second communication different from the first communication,
wherein the first communication comprises at least one of Bluetooth, BLE, and WiFi communication, and the second communication comprises at least one of UWB and IrDA communication.

5. The electronic device of claim 1, wherein the electronic device comprises a wearable device, the wearable device selected from a group consisting of a watch, ring, and ear phone.

6. A method in an electronic device, the method comprising:
measuring a body temperature of a user through a sensor module of the electronic device, thereby resulting in a measured body temperature;
estimating a core body temperature of the user, based on the measured body temperature; and
providing body temperature information of the user, based on the core body temperature,
wherein the measured body temperature comprises a shell temperature of a body part making contact with the electronic device; and
wherein the core body temperature comprises the core body temperature of an internal organ of a body of the user.

7. The method of claim 6, wherein estimating of the core body temperature comprises estimating the core body temperature by correcting the measured body temperature, based on a predefined body temperature correction value for a body part on which the electronic device is worn.

8. The method of claim 6, wherein providing of the body temperature information comprises:
detecting a predetermined event related to provision of the body temperature information, and
providing body temperature information to an external device through a first communication module configured for a first communication or a second communication module configured for a second communication different from the first communication,
wherein the first communication comprises at least one of Bluetooth, BLE, and WiFi communication, and the second communication comprises at least one of UWB and IrDA communication.

9. An electronic device comprising:
a communication module;
a display module; and
a processor operatively connected to the communication module and the display module,
wherein the processor is configured to:
search for at least one neighboring external device in response to a user input,
provide a list, based on at least one found external device on the display module,
detect an event for acquiring body temperature information,
acquire body temperature information from a selected one of the at least one neighboring external device through the communication module, and
provide the body temperature information through the display module.

10. The electronic device of claim 9, wherein the user input comprises an input for executing a predetermined application for managing a body temperature, and the processor is configured to search for neighboring external devices, using one of a first communication module and a second communication module.

11. The electronic device of claim 10, wherein the first communication module is configured for communication using at least one of Bluetooth, BLE, and WiFi communication, and the second communication module is configured to communicate using at least one of UWB and IrDA communication.

12. The electronic device of claim 11, wherein the processor is configured to:
search for the at least one neighboring external device in a predetermined space within a range using the first communication module to designate the range, and
determine an external device of a target whose body temperature is to be measured based on determining that the external device is in a predetermined direction or distance by the second communication module.

13. The electronic device of claim 11, wherein the processor is configured to:
recognize at least one external device existing in a predetermined space using the first communication module,
designate one external device of the at least one external device as an external device for measuring a body temperature using the second communication module, and
acquire the body temperature information from a designated external device through the second communication module.

14. A method of operating an electronic device, the method comprising:
searching for at least one neighboring external device in response to a user input;
providing a list, based on at least one found external device on a display module;
detecting an event for acquiring body temperature information;
acquiring body temperature information from a selected one of the at least one neighboring external device; and
providing the body temperature information through the display module.

15. The method of claim 14, wherein acquiring of the body temperature information comprises recognizing at least one neighboring external device existing in a predetermined space within a predetermined range by a first communication module,
designating a range, designating an external device of a target whose body temperature is to be measured in a predetermined direction and/or distance among the at least one external device by a second communication module; and
designating the target whose body temperature is to be measured, and acquiring the body temperature information from a designated external device through the second communication module.
